SEQUENCE LISTING (3 sequences)

SEQ ID NO: 1 - DNA, 2269 bp, Artificial Sequence
  H2AFZ DNA sequence
attggtgggatgagcaatccgagttcccggatgagggaacattctgcagtataaagggagcagggaaggcgggagacagcgcagtttgaatcgcggtgcgacgaaggagtaggtggtgggatctcaccgtgggtccgattagccttttctctgccttgcttgcttgagcttcagcggaattcgaaatggtaaacttcgcagagacgctcgatgactccgcgggtttttgccggcgagagaaaaacccgttacacagagggatgggggtgaagtggcgacggttgggaacgcgcggagacaggatttagaagagtggggggaggtgttttgtcggctgctagcggagcagcctcggatctcgcggcgggcggcgacggttgggtggcgcgcgcagcgccgctgggggccggcgggcggaggcgcgcggccgtggcgagcgcgcggcggggggcgaacgggcggggtcgcccctgcggcgcgccgcgggccggagaccccggcagggtgcggggacgcgctcgcggcgggacgcgccgcggtgggggatgggcgcgccgccttggtaattctatattctccgttcctcctccgctcgcgggcgtgtgcgcgccgcaggctggcggtaaggctggaaaggactccggaaaggccaagacaaaggcggtttcccgctcgcagagagccggcttgcaggtcagtagttgttcgtgcagtctgggagtcatgatgtttttctttgcatttccttttttgttattcatcgctctcgatgggcgttttgtgtatgcgtgggtgatgcggtgtcgcgacttgggctgcccatacgataaatatgggggggaggaatcacgtgtttcgatgtcgggacgcgttgctgcgcccgtgccccttgctttggcgcgcatataatttccccatctctagttctcatttttgtgcatttatgtttgtgtatgcttaaaatttaagttcccagtgggccgtattcatcgacacctaaaatctaggacgaccagtcatggacgtgtgggcgcgactgccgctgtgtacagcgcagccatcctggagtacctcaccgcagaggtaaatgggtgaacgcctataatccggagaaggtttggggggcggggaggcggcaagagggaaggaggaaagtgatgcaagaaaactcccaggccctgaacgcggcgagaggcctaagagaacgctagagggagctggtgttcaacaaggatcctactgagcttgagttctctgctcttggaattaaaattgcgtgccccctgtatatcttgccagtcagatagtgaggaacgattccagttggtagatctgcgtttgtgagggttttattctgttctctgaatcttggccaaaggaaagttggaaggcaactgacagattctgccttttaggtacttgaactggcaggaaatgcatcaaaagacttaaaggtaaagcgtattacccctcgtcacttgcaacttgctattcgtggagatgaagaattggattctctcatcaaggctacaattgctggtggtggtatgttaacttctaacattttaaaaaatttcttcagaggaaggaattttttgctgcttttaattagtttttccaggagaggaaatttaagtatattttcaatgatggaagtatggttgtatcatgaaatttgatttatatgtataactcaatgaatttttacttcatacttgagctgcatgtttttaaagatacctttcaagttgaacagtatacactttcttggtttcaaatactgtgattttttaaaaaatcttaagtagaattaattcctgtcactctcctaggtgtcattccacacatccacaaatctctgattgggaagaaaggacaacagaagactgtctaaaggatgcctggattccttgttatctcaggactctaaatactctaacagctgtccagtgttggtgattccagtggactgtatctctgtgaaaaacacaattttgcctttttgtaattctatttgagcaagttggaagtttaattagctttccaaccaaccaaatttctgcattcgagtcttaaccatatttaagtgttactgtggcttcaaagaagctattgattctgaagtagtgggttttgattgagttgactgtttttaaaaaactgtttggattttaattgtgatgcagaagttatagtaacaaacatttggttttgtacagacattatttccactctggtggataagttcaataaaggtcatatcccaaac

SEQ ID NO: 2 - RNA, 22 nt, Artificial Sequence
  H2AFZ siRNA sequence
gaagaaaggacaacagaagacu

SEQ ID NO: 3 - protein, 128 aa, Artificial Sequence
  H2A.Z.1 amino acid sequence
MAGGKAGKDSGKAKTKAVSRSQRAGLQFPVGRIHRHLKSRTTSHGRVGATAAVYSAAILEYLTAEVLELAGNASKDLKVKRITPRHLQLAIRGDEELDSLIKATIAGGGVIPHIHKSLIGKKGQQKTV

(19) United States
(12) Patent Application Publication
(10) Pub. No.: US 2017/0204468 A1
(43) Pub. Date: Jul. 20, 2017

(54) USE OF H2A.Z.1 AS A HEPATOCELLULAR CARCINOMA BIOMARKER

(71) Applicant: THE CATHOLIC UNIVERSITY OF KOREA INDUSTRY-ACADEMIC COOPERATION FOUNDATION, Seoul (KR)

(72) Inventors: Suk-Woo NAM, Seoul (KR); Hee-Doo YANG, Dongjak-gu (KR)

(73) Assignee: THE CATHOLIC UNIVERSITY OF KOREA INDUSTRY-ACADEMIC COOPERATION FOUNDATION, Seoul (KR)

(21) Appl. No.: 15/408,987

(22) Filed: Jan. 18, 2017

(30) Foreign Application Priority Data
Jan. 19, 2016 (KR) 10-2016-0006638

(51) Int. Cl. C12Q 1/68; G01N 33/574; C12N 15/113

(52) U.S. Cl. CPC C12Q 1/6886; C12N 15/113; G01N 33/57438; C12Q 2600/158; C12Q 2600/118; C12Q 2600/136; C12N 2310/14; G01N 2333/47

(57) ABSTRACT

The present disclosure relates to a use of H2AFZ as a hepatocellular carcinoma (HCC) biomarker and more particularly, to a marker for diagnosing hepatocellular carcinoma consisting of a H2AFZ gene or an expression protein H2A.Z.1 thereof, a composition for diagnosing or estimating prognosis of HCC, a method for diagnosing or estimating prognosis of HCC, a method of detecting a biomarker for diagnosing or estimating prognosis of HCC, a screening method of an HCC therapeutic agent, and a pharmaceutical composition for preventing or treating HCC. As it is verified that the expression level of the H2AFZ gene according to the present disclosure is increased in an HCC tissue or HCC cells compared to a non-HCC tissue or non-HCC cells, in the case of using the H2AFZ gene as the marker for diagnosing the HCC, the HCC can be rapidly and accurately diagnosed and predicted in early stages and the H2AFZ gene can be used as a target for developing a therapeutic agent for preventing or treating the HCC.

### CROSS-REFERENCE TO RELATED APPLICATIONS

This application is based on and claims priority from Korean Patent Application No. 10-2016-0006638, filed on Jan. 19, 2016, with the Korean Intellectual Property Office, the disclosure of which is incorporated herein in its entirety by reference.

### TECHNICAL FIELD

The present disclosure relates to a use of H2A.Z.1 as a hepatocellular carcinoma (HCC) biomarker and more particularly, to a novel composition for diagnosing or estimating prognosis of HCC capable of efficiently diagnosing and predicting the HCC in early stage, a method for diagnosing or estimating prognosis of HCC, a method of detecting a biomarker for diagnosing or estimating prognosis of HCC, a screening method of an HCC therapeutic agent, and a pharmaceutical composition for preventing or treating HCC.

### BACKGROUND

Hepatocellular carcinoma (HCC) is one of the most fatal cancers in the world. Particularly, in Asia and sub-Saharan Africa, more than about half a million people have died from the HCC every year. Even if a risk factor of the HCC is stable infection by hepatitis B or hepatitis C virus, a molecular mechanism in HCC cells is still not clearly defined.

Currently, for HCC diagnosis, ultrasonography, imaging diagnosis tests such as CT scanning and MRI scanning, a serum test (AFP, PIVKA-II), and a pathologic tissue test of biopsy materials have been used, and the serum AFP test has a high value at approximately less than 70% of patients, but even in the patients with chronic liver diseases, it is often recognized that a high value of severity is represented, and thus discrimination thereof is important. Meanwhile, in the early HCC, in many cases, a low value is represented, and a positive rate of protein induced by vitamin K antagonist II (PIVKA-II) is as low as less than 50%, but it is known that specificity for HCC is high and diagnosis accuracy is increased by a combination thereof. However, development of a more specific tumor marker for false positive or quantum negative cases has been expected.

A pathology tissue of the biopsy materials is an important test for the definitive diagnosis of liver diseases. However, by only a pathology feature, it is difficult to distinguish a non-cancer tissue from a cancer, particularly, early HCC. That is, a small lesion may be tubercle and early high-differential type hepatocellular carcinoma, and particularly, in the case of extracting a tumorous tissue by transepithelial biopsy specimens, an extracted amount is limited and thus a more accurate diagnosis technology is required. Accordingly, development of a cancer-specific antibody or marker which may distinguish the early hepatocellular carcinoma from the non-cancer tissue is required.

Meanwhile, the genome DNA in the eukaryotic cell is packed in the chromatin together with nucleosome including canonical histone units of H2A, H2B, H3, H4 and H1. The histones are constituted by binding the nucleosome with H3-H4 heterodimer and H2A-H2B heterodimer by a linker histone H1. A very rapid change of nucleosome compositions and biochemical properties can regulate transcription, gene slicing, DNA replication and recombination. H2A.Z is one of histone H2A modifiers having about 60% identity with the canonical histone H2A in the mammal cells and associated with chromatin reconstitution, gene transcription regulation, chromosome segregation and DNA repair. H2A.Z exchange is promoted by an ATP-dependent exchange factor including a Snf2-Related CREBBP activator (SRCAP) and a p400/Tip60 complex and this plays an important role in important biological processes such as chromosome segregation, cell cycle progression and maintenance of a heterogeneous chromatin/euchromatin state. It is known that the histones are necessary in many living organisms, but in invertebrates, only one gene copy exists and in vertebrates, two different gene copies exist. It is known that these gene copies consist of two functionally different proteins, that is, H2A.Z.1 encoded by H2AFZ and H2A.Z.2 encoded by H2AFV and recently, the genes are associated with various cancers. The H2A.Z.1 and the H2A.Z.2 have only three different amino acids at a protein level, but are encoded by different base sequences. Currently, a lot of parts of a specific function of each isoform are not yet defined, but in an H2A.Z.1 knock-down mouse study, the two genes are necessary, and further, according to a preliminary data, it is known that the genes have structural and functional differences of the nucleosomes (Non-Patent Document 2). For example, in xenograft tumors (androgen-uninvolved tumor) and castration-resistant lymph node carcinoma, a marked increase of H2A.Z.1 was observed (Non-Patent Document 3).

However, many studies on structural and functional differences between two isoforms of the H2A.Z are required, and further, in terms of an effect of H2A.Z on tumor formation, it is reported that the H2A.Z is overexpressed in breast cancer, testicular cancer, and bladder cancer, but in this case, the H2A.Z.1 is focused alone or a difference between the two isoforms is not distinguished. Accordingly, more efficient and more specific anticancer biomarkers are required by considering the specific difference between the two important isoforms.

### SUMMARY

The present disclosure has been made in an effort to provide an effective biomarker capable of rapidly and accurately diagnosing hepatocellular carcinoma (HCC) in early stage in development and progression of the HCC. Further, the present disclosure has been made in an effort to provide a composition for diagnosing HCC including a material of measuring level of an HCC-specific gene or level of a protein thereof. Further, the present disclosure has been made in an effort to provide a kit for diagnosing HCC including the composition for diagnosing the HCC of the present disclosure, a method of predicting and diagnosing HCC, and a method of screening a material capable of preventing or treating HCC. Further, the present disclosure has been made in an effort to provide a pharmaceutical composition for preventing or treating HCC including a material of inhibiting expressing of an HCC-specific gene.

In aspect of the present disclosure, there is provided a marker for diagnosing hepatocellular carcinoma (HCC) consisting of an H2AFZ gene or an H2A.Z.1 protein encoded from the gene.

The H2AFZ gene of the marker for diagnosing the HCC may have a base sequence of SEQ ID NO: 1.

In an aspect of the present disclosure, there is provided a composition for diagnosing or estimating prognosis of HCC including a material for measuring an expression level of the H2AFZ gene.

The material for measuring the expression level of the gene in the composition for diagnosing or estimating prognosis of HCC may be a material for detecting any one or more of presence, amount, and abundance pattern of mRNA transcribed by the gene and/or a protein encoded by the gene.

The material for measuring the expression level of the gene in the composition for diagnosing or estimating prognosis of HCC may be at least one of a primer, a probe, an aptamer, and antisense which are specifically bound to at least one selected from the group consisting of a nucleotide sequence of the gene, a complementary sequence thereof, a fragment of the nucleotide and a complementary sequence thereof.

The material for measuring the expression level of the gene in the composition for diagnosing or estimating prognosis of HCC may be at least one selected from oligopeptides, monoclonal antibodies, polyclonal antibodies, chimeric antibodies, antibody fragments, ligands, peptide nucleic acids (PNA), aptamers, avidity multimers, and peptidomimetics which specifically bind to at least one of a polypeptide encoded by a nucleotide sequence of the gene, a polypeptide encoded by a complementary sequence thereto, and a polypeptide encoded by a fragment of the nucleotide sequence.

The material for measuring the expression level of the gene in the composition for diagnosing or estimating prognosis of HCC may be a detection reagent of measuring a gene expression by at least one method of a reverse transcription polymerase chain reaction, a competitive polymerase chain reaction, a real-time polymerase chain reaction, a nuclease protection assay (RNase, S1 nuclease assay), an in situ hybridization method, a DNA microarray method, northern blotting, western blotting, an enzyme linked immuno sorbent assay (ELISA), a radioimmunoassay, an immunodiffusion method, immunoelectrophoresis, a tissue immuno staining, an immunoprecipitation assay, a complement fixation assay, an FACS, a mass spectrometry and a protein microarray.

In an aspect of the present disclosure, there is provided a kit for diagnosing or estimating prognosis of HCC including the composition for diagnosing or estimating prognosis of the HCC.

The kit may be at least one selected from a microarray, a gene amplification kit, an immunoassay kit, a luminex assay kit, a protein microarray kit, and an ELISA kit.

In an aspect of the present disclosure, there is provided a method for diagnosing or estimating prognosis of the HCC including: treating a target biological sample with the composition for diagnosing or estimating prognosis of the HCC; measuring an expression level of the H2AFZ gene from the target biological sample; and comparing a measured result of the gene expression level with a reference value.

The biological sample may be selected from the group consisting of tissues, cells, blood, serum, plasma, saliva and urine.

The measuring of the expression level of the gene in the method for diagnosing or estimating prognosis of HCC may use at least one method of a reverse transcription polymerase chain reaction, a competitive polymerase chain reaction, a real-time polymerase chain reaction, a nuclease protection assay (RNase, S1 nuclease assay), an in situ hybridization method, a DNA microarray method, northern blotting, western blotting, an enzyme linked immuno sorbent assay (ELISA), a radioimmunoassay, an immunodiffusion method, immunoelectrophoresis, a tissue immuno staining, an immunoprecipitation assay, a complement fixation assay, an FACS, a mass spectrometry and a protein microarray.

In an aspect of the present disclosure, there is provided a method of detecting a biomarker for diagnosing or estimating prognosis of the HCC through measuring an expression level of the H2AFZ gene in the human biological sample in order to provide information required for diagnosing or estimating prognosis of the HCC.

The measuring of the expression level of the gene in the method of detecting a biomarker for diagnosing or estimating prognosis of the HCC may use at least one method of a reverse transcription polymerase chain reaction, a competitive polymerase chain reaction, a real-time polymerase chain reaction, a nuclease protection assay (RNase, S1 nuclease assay), an in situ hybridization method, a DNA microarray method, northern blotting, western blotting, an enzyme linked immuno sorbent assay (ELISA), a radioimmunoassay, an immunodiffusion method, immunoelectrophoresis, a tissue immuno staining, an immunoprecipitation assay, a complement fixation assay, an FACS, a mass spectrometry and a protein microarray.

In an aspect of the present disclosure, there is provided a screening method of an HCC therapeutic agent including verifying whether a test target compound promotes or inhibits expression of the H2AFZ gene.

The verifying of whether to promote or inhibit the expression of the gene in the screening method of an HCC therapeutic agent may use at least one method of a reverse transcription polymerase chain reaction, a competitive polymerase chain reaction, a real-time polymerase chain reaction, a nuclease protection assay (RNase, S1 nuclease assay), an in situ hybridization method, a DNA microarray method, northern blotting, western blotting, an enzyme linked immuno sorbent assay (ELISA), a radioimmunoassay, an immunodiffusion method, immunoelectrophoresis, a tissue immuno staining, an immunoprecipitation assay, a complement fixation assay, an FACS, a mass spectrometry and a protein microarray.

In an aspect of the present disclosure, there is provided a pharmaceutical composition for preventing or treating HCC including, as an active ingredient, an antisense or small interference RNA (siRNA) oligonucleotide having a complementary sequence to a base sequence of a H2AFZ gene.

The siRNA oligonucleotide using in the pharmaceutical composition for preventing or treating HCC may consist of a base sequence of SEQ ID NO: 2.

According to the exemplary embodiments of the present disclosure, as it is verified that the expression level of the H2AFZ gene according to the present disclosure is increased in an HCC tissue or HCC cells compared to a non-HCC tissue or non-HCC cells, in the case of using the H2AFZ gene as the marker for diagnosing the HCC, the HCC can be rapidly and accurately diagnosed and predicted in early stages and the H2AFZ gene can be used as a target for developing a therapeutic agent for preventing or treating the HCC.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIGS. 1A-1F are diagram of various experimental results verifying that H2A.Z.1 is overexpressed in human hepatocellular carcinoma cells (HCC).

FIG. 1A is a microarray analysis result from Gene Expression Omnibus (GEO) database (GSE14520, GSE16757, GSE22058 and GSE36376). (Mean±standard deviation, ***P<0.001, versus non-tumor).

FIG. 1B is a result of qRT-PCR performing expression of H2AFZ mRNA in human HCC cells.

FIG. 1C is a western blot result (T; tumor tissue, N: non-tumor tissue) for H2AFZ in the human HCC cells, in which glyceraldehyde-3-phosphate dehydrogenase (GAPDH) is used as a loading control.

FIG. 1D is a result of performing qRT-PCR for one normal hepatocyte (MIHA) and nine HCC cell lines (HepG2, Hep3B, Huh7, PLC/PRF/5, SK-Hep1, SNU-182, SNU-368, SNU-449, and SNU-475). Glyceraldehyde-3-phosphate dehydrogenase (GAPDH) is a control (Mean±standard deviation, n=3, *P<0.05, **P<0.01, versus MIHA cells).

FIG. 1E is a western blot result of expression of an endogenous protein H2A.Z.1 in liver cancer cell lines. Glyceraldehyde-3-phosphate dehydrogenase (GAPDH) is a loading control.

FIG. 1F is a microarray analysis result from Gene Expression Omnibus (GEO) database of mice (GSE29813, GSE35289, and GSE57597). (Mean±standard deviation, ***P<0.001, versus non-tumor).

FIGS. 2A-2D are clinical relation of H2AFZ expression in an HCC patient and an analysis result of H2AFZ-related gene.

FIG. 2A is microarray data obtained from a GEO database (GSE16757). Cluster analysis is performed by selecting 524 genes (P<0.01, r>0.4 or r<−0.4). Patients are divided into a high-H2AFZ group and a low-H2AFZ group.

FIG. 2B is a Kaplan-Meier survival curve for mean survivors.

FIG. 2C is a result processed with a molecular function mining tool (DAVID). A bar graph illustrates a gene enrichment score.

FIG. 2D is a GSEA analysis result for H2AFZ-specific molecular indication.

FIGS. 3A-3D are result of describing an effect of H2AFZ knock-down on cell growth and division.

FIG. 3A illustrates that SNU-449 and SK-Hep1 cell lines are infected with negative control siRNA (N.C. 100 nM) and H2A.Z.1-specific siRNA (siH2A.Z.1, 100 nM) and measured after 72 hrs and the cell number is measured by counting trypsin blue cells (Mean±standard deviation, n=3, **P<0.01).

FIG. 3B shows that a growth rate of living cells is suppressed during H2A.Z.1 knock-down. The cell growth is measured for 96 hrs. All experiments are performed three times (Mean±standard deviation, n=3, **P<0.01, ***P<0.001).

FIG. 3C is a result of bromodeoxyuridine (BrdU) incorporation assays. (Mean±standard deviation, n=3, *P<0.05)

FIG. 3D is a result of clonogenic assays. Upper panel: Representative image, lower panel: Three randomly selected (Mean±standard deviation, n=3, **P<0.01, ***P<0.001).

FIGS. 4A-4D are result of verifying that H2A.Z.1-specific destruction causes apoptosis and cell cycle arrest in HCC cells.

FIG. 4A is an analysis result of apoptosis. Negative control siRNA (N.C) or H2A.Z.1 siRNA (siH2A.Z.1) is treated in SNU-449 and SK-Hep1 cell lines. After 72 hrs, a fluorescence-activated cell sorting analysis (FACS) is performed by using PI(FL3-H) and annexin V(FL1-H). A bar graph illustrates a ratio of annexin V positive cells. (Mean±standard deviation, n=3, *P<0.001).

FIG. 4B is an analysis result of cell cycles. FACS analysis was performed after negative control siRNA or H2A.Z.1 siRNA infection. During H2A.Z.1 knock-down, arrest in a G1/S cycle occurs. % illustrates distribution of cells (Mean±standard deviation, n=3, *P<0.05).

FIG. 4C is a western blot result for caspase-3, cleaved caspase-3, PARP, and cleaved PARP. During H2A.Z.1 knock-down, apoptosis is caused in SNU-449 and SK-Hep1 cell lines. GAPDH is used as a loading control.

FIG. 4D is a western blot result. Negative control siRNA or H2A.Z.1 siRNA is treated in SNU-449 and SK-Hep1 cell lines. a protein level of H2A.Z.1, cyclin D1, p21, p2′7, CDK2, and phosphorylated-pRb (p-pRb) are measured by using antibodies. GAPDH is used as a loading control.

FIGS. 5A-5E illustrate results of verifying that H2A.Z.1 expression inhibition suppresses metastasis potential of HCC cells.

FIG. 5A verifies of suppressing in vitro cell migration and invasion of SNU-449 and SK-Hep1 cell lines during H2A.Z.1 knock-down. A graph is illustrated by measuring the number of migrated and invaded cells and performed an experiment three times (Mean±standard deviation, n=3, **P<0.01, ***P<0.001). The drawing is a representative image.

FIG. 5B illustrates a scratch wound healing assay result. Cells are scratched and wounded and incubated for 24 hours. The rod graph is a ratio of restored area (mean±standard deviation, n=3, ***P<0.001, *P<0.05).

FIG. 5C is a western blot result for an epithelial mesenchymal transition (EMT) marker. During H2A.Z.1 knock-down, EMT proteins are selectively changed. The EMT proteins are detected by using specific antibodies for H2A.Z.1, E-cadherin, fibronectin, N-cadherin, vimentin, snail and slug. GAPDH is used as a loading control.

FIG. 5D analyzes microarray data from a gene expression omnibus (GEO) database. For analyzing expression between H2AFZ and FN1, GEO data of accession numbers GSE36376 are used. A left panel illustrates FN1 mRNA expression in GSE36376 and a right panel illustrates a quantitative relation between H2AFZ and FN1 in GSE36376 (Pearson correlation coefficient r=0.4273, ***P<0.0001).

FIG. 5E is a result of analyzing a network related to specific molecular indication of H2AFZ, in which H2AFZ-related genes are analyzed by using a web tool called a concept map.

### DETAILED DESCRIPTION

In the following detailed description, reference is made to the accompanying drawing, which forms a part hereof. The illustrative embodiments described in the detailed description, drawing, and claims are not meant to be limiting. Other embodiments may be utilized, and other changes may be made, without departing from the spirit or scope of the subject matter presented here.

The present disclosure relates to a use of H2AFZ as a hepatocellular carcinoma (HCC) biomarker and more particularly, to a marker for diagnosing hepatocellular carcinoma consisting of a H2AFZ gene or an expression protein H2A.Z.1 thereof, a composition for diagnosing or estimating prognosis of HCC, a method for diagnosing or estimating prognosis of HCC, a method of detecting a biomarker for diagnosing or estimating prognosis of HCC, a screening method of an HCC therapeutic agent, and a pharmaceutical composition for preventing or treating HCC. Hereinafter, the present disclosure will be described in detail with reference to the drawings.

The inventors studied a novel biomarker that can quickly and accurately diagnose HCC and estimate prognosis, defined that the biomarkers of the present disclosure can diagnose HCC in early stage and determine prognosis, and completed the present disclosure.

In the present disclosure, the term “liver cancer” generally means a cancer derived from hepatocytes. The liver cancer includes a primary liver cancer derived from the liver from the beginning and a metastatic liver cancer caused by transiting the cancer generated from other tissues to the liver. Most of causes are unclear, but cirrhosis is often present, and the liver cancer has been found to occur in patients with liver cirrhosis, chronic active hepatitis B or hepatitis B carriers. The inventors may obtain a high result having high sensitivity and reliability for occurrence of the liver cancer from the subject.

In this specification, the term of diagnosis includes determining susceptibility of one subject for a specific disease or disorder, determining whether one subject has the specific disease or disorder at present, determining prognosis of one subject having the specific disease or disorder (for example, identification of pre-metastatic or metastatic cancerous conditions, determination of the stage of cancer or determination of the reactivity of cancer to treatment), or therametrics (for example, monitoring an subject state in order to provide information on the therapeutic efficacy). The “prognosis” of the liver cancer may be estimated in various aspects, but representatively determined in terms of a recurrence possibility, a survival possibility, and a disease free survival possibility.

In this specification, the term “the (bio)marker, and the marker or the diagnosis marker for diagnosis” is a material capable of distinguishing and determining cells or tissues with the liver cancer from normal cells or tissues and includes organic biomolecules such as a polypeptide or a nucleic acid (for example, mRNA and the like), a lipid, a glycolipid, a glycoprotein, and sugars (monosaccharide, disaccharide, oligosaccharide, and the like) which are increased in cells with the liver cancer compared to the normal cells. For the purpose of the present disclosure, the (bio)marker for diagnosing the HCC is a nucleotide (including segments thereof) of a H2AFZ gene or a protein (including segments thereof) encoded thereon and a gene with increased expression in the HCC cells. The markers may use mRNA for any one gene or any one protein encoded by the gene and may be complex markers including two or more markers.

It is interpreted that the ‘polypeptide (alternatively, the protein)’ used in this specification includes amino acid sequence having substantial identity to the corresponding amino acid sequence. The substantial identity means an amino acid sequence having at least 60% homology, more preferably at least 80% homology, and most preferably at least 90% homology, in the case of analyzing a sequence aligned to maximally correspond to any different sequence to the amino acid sequence of the present disclosure and aligned by using a generally used algorithm.

For example, the polypeptide (protein) includes a polypeptide having an amino acid sequence with identity of approximately 60% or more, 80% or more, 90% or more, 95% or more, 99% or more, 99.1% or more, 99.2% or more, 99.3% or more, 99.4% or more, 99.5% or more, or 99.9% or more to the specific amino acid sequence and functioning as a biomarker for diagnosing or estimating prognosis of the HCC. Generally, it is more preferred that identity % is increased.

Further, the polypeptide having the identity includes a polypeptide related with a beta-adipate pathway while including an amino acid sequence in which one or more amino acid residues are deleted, substituted, inserted, and/or added in the polypeptide having a specific amino acid sequence. Generally, it is more preferred that the deleted, substituted, inserted, and/or added number is decreased.

The ‘polynucleotide’ (alternatively, nucleotides and nucleic acids) used in this specification has a meaning comprehensively including DNA (gDNA and cDNA) and RNA molecules and the nucleotide as a basic constitute unit in the nucleic acid molecule includes analogues with a modified sugar or base site as well as a natural nucleotide.

It is interpreted that the polynucleotide of the present disclosure is not limited to nucleic acid molecules encoding the specific amino acid sequence (polypeptide) and includes nucleic acid molecules encoding an amino acid sequence having substantial identity to the specific amino acid sequence or polynucleotide having a function corresponding thereto as described above. The substantial identity means an amino acid sequence having at least 60% homology, more preferably at least 80% homology, and most preferably at least 90% homology, in the case of analyzing a sequence aligned to maximally correspond to any different sequence to the amino acid sequence of the present disclosure and aligned by using a generally used algorithm.

The polypeptide having the corresponding function includes for example, a polypeptide of an amino acid sequence in which one or more amino acids are deleted, substituted, inserted, and/or added. The polypeptide consists of an amino acid sequence in which one or more amino acid residues are deleted, substituted, inserted, and/or added and includes a polypeptide involved in synthesis of 3-hydroxypropionic acid as described above and it is preferred that the number of deleted, substituted, inserted, and/or added amino acid residues is small. Further, the polypeptide has an amino acid sequence having about 60% or more of identity to the specific amino acid sequence as described above and includes a polypeptide having a biomarker function for diagnosing or estimating prognosis of the HCC, and high identity is preferable.

The term “complementary” or “complementarity” used in this specification means a function of forming a double strand polynucleotide by coupling purine and pyrimidine nucleotides through hydrogen bonding and includes partially complementary cases. The following base pairs are associated with complementarity: Guanine and cytosine; adenine and thymine; and adenine and uracil. The “complementary” is substantially applied to all base pairs including two single-strand polynucleotides over the total length of molecules in the aforementioned relationship. The “partially complementary” means a relationship in which a part of one of the molecules remains a single strand because a length of one of the two single-strand polynucleotides is short.

The present disclosure provides a marker for diagnosing hepatocellular carcinoma (HCC) consisting of an H2AFZ gene or an H2A.Z.1 protein encoded from the gene. The H2AFZ gene of the marker for diagnosing the HCC may have a base sequence of SEQ ID NO: 1.

The present disclosure provides a composition for diagnosing or estimating prognosis of HCC including a material for measuring an expression level of the H2AFZ gene.

In the composition for diagnosing or estimating prognosis of the HCC, the material for measuring the expression level of the gene may detect at least one of presence, amount, and abundance pattern of mRNA transcribed by the gene and/or a protein encoded by the gene.

In the present disclosure, the level of the H2AFZ gene means preferably an mRNA level expressed by the H2AFZ gene, that is, an amount of the mRNA and may include a specific primer or probe for the H2AFZ gene as a material capable of measuring the level. In the present disclosure, the specific primer or probe to the H2AFZ gene may be a primer or probe which may specifically amplify the entire H2AFZ gene represented by SEQ ID NO: 1 or a specific region of the gene, and the primer or probe may be designed by a known method in the art.

In the present disclosure, the term of the primer means a single-strand oligonucleotide capable of acting as an initial point of template-directed DNA synthesis under a suitable condition (that is, four different nucleoside triphosphates and polymerases) at a suitable temperature and in a suitable buffer solution. The suitable length of the primer may be changed according to various elements, for example, a temperature and a use of the primer. Further, the sequence of the primer needs not to have a completely complementary sequence to a partial sequence of the template and is hybridized with the template to have suitable complementarity within the range of the primer-specific action. Accordingly, the primer in the present disclosure needs not to have a completely complementary sequence to the nucleotide sequence of the H2AFZ gene as the template and is hybridized with the gene sequence to have suitable complementarity within the range capable of acting the primer. Further, the primer according to the present disclosure may be used for a gene amplification reaction.

In the present disclosure, the term of probe means a natural or modified monomer or a linkage ([0046] linkages) linear oligomer and includes deoxyribonucleotide and ribonucleotide, and can specifically hybridize to a target nucleotide sequence, and is naturally present or artificially synthesized. The probe according to the present disclosure may be a single chain and preferably, may be oligodeoxyribonucleotide. The probe of the present disclosure may include natural dNMPs (i.e., dAMP, dGMP, dCMP, and dTMP), nucleotide analogs or derivatives. Further, the probe of the present disclosure may include ribonucleotide. For example, the probe of the present disclosure may include skeleton modified nucleotides, such as peptide nucleic acids (PNA) (M. Egholm et al., Nature, 365: 566-568 (1993)), phosphorothioate DNA, phosphorodithioate DNA, phosphoamidate DNA, amide-linked DNA, MMI-linked DNA, 2′-O-methyl RNA, alpha-DNA and methylphosphonate DNA, sugar modified nucleotides, such as 2′-O-methyl RNA, 2′-fluoro RNA, 2′-amino RNA, 2′-O-alkyl DNA, 2′-O-allyl DNA, 2′-O-alkynyl DNA, hexose DNA, pyranosyl RNA and anhydrohexitol DNA, and base-modified nucleotides, such as C-5 substituted pyrimidines (the substituents include fluoro-, bromo-, chloro-, iodo-, methyl-, ethyl-, vinyl-, formyl-, ethytyl-, propynyl-, alkanyl-, thiazolyl-, imidazolyl-, and pyridyl-), 7-deazapurines with C-7 substituents (the substituents include fluoro-, bromo-, chloro-, iodo-, methyl-, ethyl-, vinyl-, formyl-, alkanyl-, alkenyl-, thiazolyl-, imidazolyl-, and pyridyl-), inosine and diaminopurine.

Accordingly, in the composition for diagnosing or estimating prognosis of the HCC, the material for measuring the expression level of the gene may be at least one of a primer, a probe, an aptamer, and antisense which are specifically bound to at least one selected from the group consisting of a nucleotide sequence of the gene, a complementary sequence thereof, a fragment of the nucleotide and a complementary sequence thereof.

Further, in the present disclosure, the level of the H2A.Z.1 protein expressed by the H2AFZ gene means preferably an H2A.Z.1 polypeptide generated through a translation process from mRNA expressed by the H2AFZ gene and the sequence of the H2A.Z.1 polypeptide is represented by SEQ ID NO: 3.

The material capable of measuring the level of the H2A.Z.1 protein may include antibodies such as polyclonal antibodies, monoclonal antibodies, and recombinant antibodies capable of specifically binding to the H2A.Z.1 protein. Accordingly, in the composition for diagnosing or estimating prognosis of the HCC, the material for measuring the expression level of the gene may be at least one selected from oligopeptides, monoclonal antibodies, polyclonal antibodies, chimeric antibodies, antibody fragments, ligands, peptide nucleic acids (PNA), aptamers, avidity multimers, and peptidomimetics which specifically bind to at least one of a polypeptide encoded by a nucleotide sequence of the gene, a polypeptide encoded by a complementary sequence thereto, and a polypeptide encoded by a fragment of the nucleotide sequence.

In the present disclosure, as described above, since the H2A.Z.1 protein is defined as the marker protein capable of diagnosing the HCC, the method of generating the antibody using the protein may be easily prepared using a technique known to those skilled in the art. For example, the polyclonal antibodies may be produced by a well-known method in the art of injecting H2AFZ(H2A.Z.1) antigens to an animal and obtaining the blood from the animal to obtain the serum including the antibodies and the polyclonal antibodies may be prepared from any animal species host such as goat, rabbit, sheep, monkey, horse, pig, cattle, and dog. The monoclonal antibodies may be prepared by using a hybridoma method (Kohler et al., European Journal of Immunology, 6, 511-519, 1976) well-known in the art or prepared by using a phage antibody library technique (Clackson et al, Nature, 352, 624-628, 1991, Marks et al, J. Mol. Biol., 222:58, 1-597, 1991). Further, the antibodies according to the present disclosure may include functional fragments of antibody molecules as well as a complete form with two full length light chains and two full length heavy chains. The functional fragment of the antibody molecule means a fragment having at least antigen binding function and includes Fab, F(ab′), F(ab′) 2 and Fv.

In the composition for diagnosing or estimating prognosis of the HCC, the material for measuring the expression level of the gene may be a detection reagent of measuring a gene expression by at least one method of a reverse transcription polymerase chain reaction, a competitive polymerase chain reaction, a real-time polymerase chain reaction, a nuclease protection assay (RNase, S1 nuclease assay), an in situ hybridization method, a DNA microarray method, northern blotting, western blotting, an enzyme linked immuno sorbent assay (ELISA), a radioimmunoassay, an immunodiffusion method, immunoelectrophoresis, a tissue immuno staining, an immunoprecipitation assay, a complement fixation assay, an FACS, a mass spectrometry and a protein microarray.

The present disclosure a kit for diagnosing or estimating prognosis of the HCC including the composition for diagnosing or estimating prognosis of the HCC. The kit may be at least one selected from a microarray, a gene amplification kit, an immunoassay kit, a luminex assay kit, a protein microarray kit, and an ELISA kit.

The present disclosure provides a method for diagnosing or estimating prognosis of the HCC including: treating a target biological sample with the composition for diagnosing or estimating prognosis of the HCC; measuring an expression level of the H2AFZ gene from the target biological sample; and comparing a measured result of the gene expression level with a reference value. The biological sample may be selected from the group consisting of tissues, cells, blood, serum, plasma, saliva and urine.

The expression level of the H2AFZ gene preferably measures an mRNA level and the method of measuring the mRNA level includes a reverse transcription polymerase chain reaction (RT-PCR), a real-time reverse transcriptase chain reaction, an RNase protection assay, a northern blot, a DNA chip, and the like, but is not limited thereto. In the method for diagnosing or estimating prognosis of the HCC, the measuring of the expression level of the gene may use at least one method of a reverse transcription polymerase chain reaction, a competitive polymerase chain reaction, a real-time polymerase chain reaction, a nuclease protection assay (RNase, S1 nuclease assay), an in situ hybridization method, a DNA microarray method, northern blotting, western blotting, an enzyme linked immuno sorbent assay (ELISA), a radioimmunoassay, an immunodiffusion method, immunoelectrophoresis, a tissue immuno staining, an immunoprecipitation assay, a complement fixation assay, an FACS, a mass spectrometry and a protein microarray.

The measuring of the H2AFZ protein level can use an antibody and in this case, a H2AFZ marker protein in the biological sample and a specific antibody thereto form a binding substance, that is, an antigen-antibody complex, and a amount of the antigen-antibody complex may be quantitatively measured by a size of signal of a detection label. The detection label may be selected from the group consisting of enzymes, minerals, ligands, emitters, microparticles, redox molecules and radioisotopes and is not limited thereto. An analysis method for measuring the protein level is not limited thereto, but includes western blot, ELISA, radioimmunoassay, radioimmunodiffusion, Ouchterlony immunodiffusion, rocket immunoelectrophoresis, tissue immuno staining, immunoprecipitation assay, complement fixation assay, FACS, protein chip, and the like.

Accordingly, in the present disclosure, through the detection methods, a H2AFZ mRNA expression amount or a protein amount in a control group and a H2AFZ mRNA expression amount or a protein amount in an HCC patient or an HCC suspicious patient may be verified, and occurrence, progression, or prognosis of the HCC may be predicted and diagnosed by comparing the degree of the expression amount of HCC patient or an HCC suspicious patient with that of the control group, that is, a normal sample.

For example, tissues and HCC cells obtained from the HCC patient are lysed to obtain a lysate including intracellular proteins, the H2AFZ protein amount is measured from each HCC sample by performing a western blot method using an antibody for H2AFZ(H2A.Z.1), and then the measured value is performed through a process of comparing and analyzing with the normal control group. Furthermore, the inventors predicted that through the above result, when the HCC occurs, the expression of the H2AFZ is increased in the cell or tissue, and thus in the case of inhibiting the expression of the H2AFZ in the HCC cells, the HCC may be prevented or treated and determined that the material capable of inhibiting the expression of the H2AFZ may be used as an HCC therapeutic agent.

The present disclosure provides a method of detecting a biomarker for diagnosing or estimating prognosis of the HCC through measuring an expression level of the H2AFZ gene in the human biological sample in order to provide information required for diagnosing or estimating prognosis of the HCC.

In the method of detecting the biomarker for diagnosing or estimating prognosis of the HCC, the measuring of the expression level of the gene may use at least one method of a reverse transcription polymerase chain reaction, a competitive polymerase chain reaction, a real-time polymerase chain reaction, a nuclease protection assay (RNase, S1 nuclease assay), an in situ hybridization method, a DNA microarray method, northern blotting, western blotting, an enzyme linked immuno sorbent assay (ELISA), a radioimmunoassay, an immunodiffusion method, immunoelectrophoresis, a tissue immuno staining, an immunoprecipitation assay, a complement fixation assay, an FACS, a mass spectrometry and a protein microarray.

The present disclosure provides a screening method of an HCC therapeutic agent including verifying whether a test target compound promotes or inhibits expression of the H2AFZ gene.

According to the method of the present disclosure, first, a sample to be analyzed may be contacted with cells including or expressing the H2AFZ gene or the H2A.Z.1 protein. Here, the sample means an unknown material used in screening in order to examine whether to affect an expression amount of the H2AFZ gene and an amount or activity of the H2A.Z.1 protein. The sample may include a chemical material, nucleotide, antisense-RNA, small interference RNA (siRNA), and a natural extract, but is not limited thereto. Thereafter, in the cells treated with the sample, the expression amount of the H2AFZ gene and the amount or activity of the H2A.Z.1 protein may be measured, and as a result, when the expression amount of the H2AFZ gene and the amount or activity of the H2A.Z.1 protein are decreased, the sample may be determined as a material for treating or preventing the HCC.

In the method of measuring the expression amount of the H2AFZ gene and the amount or activity of the H2A.Z.1 protein (whether to promote or inhibit the expression of the gene) may be performed through various methods known in the art, and for example, may use at least one method of a reverse transcription polymerase chain reaction, a competitive polymerase chain reaction, a real-time polymerase chain reaction, a nuclease protection assay (RNase, S1 nuclease assay), an in situ hybridization method, a DNA microarray method, northern blotting, western blotting, an enzyme linked immuno sorbent assay (ELISA), a radioimmunoassay, an immunodiffusion method, immunoelectrophoresis, a tissue immuno staining, an immunoprecipitation assay, a complement fixation assay, an FACS, a mass spectrometry and a protein microarray.

Further, the present disclosure provides a pharmaceutical composition for preventing or treating HCC including an antisense or siRNA oligonucleotide having a complementary sequence to a base sequence of the H2AFZ gene. The siRNA oligonucleotide used in the pharmaceutical composition for preventing or treating the HCC may consist of a base sequence of SEQ ID NO: 2.

According to the exemplary embodiment of the present disclosure, actually, in the case of inhibiting the expression of the H2AFZ gene or inhibiting the expression of the H2A.Z.1 protein, an experiment of verifying whether to prevent or treat the HCC is performed, that is, the expression of the H2AFZ is inhibited in the HCC cells using the siRNA for the H2AFZ gene, and as a result, it is exhibited that the cell growth of the HCC cells is inhibited and further, it is verified that apoptosis of the HCC cells is promoted.

Meanwhile, the normal cells maintains a balance between a self-growth and differentiation control function and a self-death function of the cells, but the number of cancer cells is exponentially and rapidly increased and grows due to the broken balance. The process of the cell cycle for growth and differentiation of the cells is largely divided into an interphase and a mitotic phase, and the interphase is constituted by a G(1)-phase where synthesis of various proteins required for cell division occurs in the cells, a S-phase where DNA synthesis occurs, and a mitosisphase which is a cell differentiation process.

Further, regulatory elements capable of regulating the cell division are required, and particularly, various cyclin proteins forms a cyclin/CDK complex with phosphorylase called cyclin dependent kinase (CDK) to regulate the steps of the cell cycle, and particularly, p21 which is cyclin kinase inhibitors (CKIs) is attached to the cyclin/CDK complex of the G1-phase to prevent the complex from being activated and inhibit progression of the cell cycle, thereby inhibiting the growth of the cells.

The cyclin protein also plays an important role in the cell cycle and synthesis and decompression of the cyclin are strictly controlled so that an expression level of the cyclin is changed. The cyclin binds to cyclin dependent serine/threonine kinase (CDK) and the binding is required in intercellular CDK (for example, CDK1 CDK2, CDK4 and/or CDK6) activity. Further, it is reported that the CDK is present downstream of a plurality of tumor gene signaling pathways, and it is known that disregulation of the CDK activity due to deletion of an upregulatory or endogenous inhibitor of the cyclin forms an important axis between the mitogen-promoting signaling pathway of tumor cells and the proliferation of tumor cells, and the inhibitor of the CDK is recognized to be useful as a selective inhibitor of cell proliferation, for example, cancer cell growth of the mammal.

As a result, the inventors expected that the H2AFZ is involved even in the cell cycle process of cancer cells through the fact that the H2AFZ is overexpressed in the HCC cells and examined a cell cycle regulator influenced by the H2AFZ. According to the result of the exemplary embodiment of the present disclosure, the gene expression of H2AFZ is inhibited by treating H2AFZ siRNA in the HCC cells and expression of cyclin D1 and CDK2 is inhibited, whereas p21, p16, p15 and cyclin D3 are not affected.

Through the result, the inventors found that the H2AFZ causes abnormality in regulation of cell cycle and growth in the cells through regulation of cyclin D1 and CDK2 to cause the cancer. Accordingly, the inventors found that the H2AFZ causes abnormality in regulation of cell cycle and growth in the cells through regulation of cyclin D1 and CDK2 to cause the cancer and further, found that in the case of inhibiting the expression of the H2AFZ in the cells, occurrence of the HCC was prevented or the HCC may be treated. Therefore, in the pharmaceutical composition for preventing or treating the HCC according to the present disclosure, all of materials capable of inhibiting the expression of the H2AFZ may be included, and preferably, chemical materials, nucleotides, antisense, siRNA oligonucleotides or natural extracts may be included as an active ingredient, more preferably, antisense or siRNA (small interference RNA) oligonucleotides having a complementary sequence to the nucleotide sequence of the H2AFZ gene of the present disclosure may be included as an active ingredient, and much more preferably, siRNA oligonucleotides for the H2AFZ gene having a base sequence of SEQ ID NO: 2 may be included.

In the present disclosure, the term “antisense oligonucleotide” means DNA or RNA or derivatives thereof including a complementary nucleic acid sequence to a sequence of a specific mRNA and binds to a complementary sequence in mRNA and acts to inhibit the translation to the H2AFZ protein. The antisense sequence of the present disclosure means a DNA or RNA sequence which is complementary to the H2AFZ mRNA and can bind to the H2AFZ mRNA and may inhibit a required activity for translation of the H2AFZ mRNA, translocation into the cytoplasma, muturation, or all other overall biological functions.

Further, the antisense nucleic acid may be deformed at a location of one or more bases, sugars or backbones in order to enhance efficiency (De Mesmaeker et al., Curr Opin Struct Biol., 5, 3, 343-55, 1995). The nucleic acid backbone may be modified by binding of phosphorothioate, phosphotriester, methylphosphonate, short-chain alkyl, cycloalkyl, short-chain heteroatomic, heterocyclic sugars. Further, the antisense nucleic acid may include one or more substituted sugar moieties. The antisense nucleic acid may include modified bases. The modified bases include hypoxanthane, 6-methyladenine, 5-methylpyrimidine (especially, 5-methylcytosine), 5-hydroxymethylcytosine (HMC), glycosyl HMC, zentobiosyl HMC, 2-aminoadenine, 2-thiouracil, 2-thiothymine, 5-bromouracil, 5-hydroxymethyluracil, 8-azaguanine, 7-deazaguanine, N6 (6-aminohexyl) adenine, 2,6-diaminopurine, and the like. Further, the antisense nucleic acid of the present disclosure may be chemically bound with one or more moieties or conjugates which improve activity and cell absorption of the antisense nucleic acid. The moieties include liposoluble moieties such as a cholesterol moiety, a cholesteryl moiety, cholic acid, thioether, thiocholesterol, an aliphatic chain, phospholipid, polyamine, a polyethylene glycol chain, adamantane acetic acid, a palmityl moiety, octadecylamine, and a hexylamino-carbonyl-oxycholesterol moiety, but are not limited thereto. Oligonucleotides including liposoluble moieties and a preparation method are well-known in the art of the present disclosure (U.S. Pat. Nos. 5,138,045, 5,218,105 and 5,459,255). The modified nucleic acid may increase stability for nuclease and increase binding affinity between antisense nucleic acid and target mRNA.

The antisense oligonucleotide may be synthesized in a test tube by a general method to be administrated in vivo or synthesized in vivo. An example of synthesizing the antisense oligonucleotide in the test tube is to use RNA polymerase I. One example of synthesizing the antisense RNA in vivo is to transcribe the antisense RNA by using a vector of which an origin of a recognition site (MCS) is in an opposite direction. It is preferred that the antisense RNA is not translated to a peptide sequence because a translation stop codon exists in the sequence.

In the present disclosure, the term “siRNA” means a nucleic acid molecule capable of mediating RNA interference or gene silencing (see International Patent Publication Nos. 00/44895, 01/36646, 99/32619, 01/29058, 99/07409 and 00/44914). The siRNA may inhibit expression of the target gene to be provided by an efficient gene knock-down method or a gene treating method.

The siRNA molecule of the present disclosure may have a double strand structure in which a sense strand (a sequence corresponding to the H2AFZ mRNA sequence) and an antisense strand (a complementary sequence to the H2AFZ mRNA sequence) are positioned to be opposite to each other and further, may have a single-chain structure having self-complementary sense and antisense strands. Further, the siRNA is not limited to complete pairing of double-stranded RNA portions that are paired with each other and a non-paired portion may be included by a mismatch (the corresponding base is not complementary), a bulge (there is no base corresponding to one chain), and the like. Further, the siRNA terminal structure includes all of blunt terminals or cohesive terminals so long as the expression of the H2AFZ gene may be inhibited by an RNAi effect and the cohesive terminal structure has both a 3′-terminal projection structure and a 5′-terminal projection structure. Further, the siRNA molecule of the present disclosure may have a form in which a short nucleotide sequence is inserted between the self-complementary sense and antisense strands, and in this case, the siRNA molecule formed by the expression of the nucleotide sequence forms a hair-pin structure by hybridization in the molecule and entirely forms a stem-and-loop structure. The stem-and-loop structure is processed in vitro or in vivo to generate an active siRNA molecule capable of mediating RNAi.

The method of preparing the siRNA includes a method of directly synthesizing siRNA in a test tube to introduce the siRNA into the cell through a transformation process and a method of transforming or infecting an siRNA expression vector or a PCR-derived siRNA expression cassette prepared so that the siRNA is expressed in the cells into the cells.

Further, the composition including the gene-specific siRNA of the present disclosure may include a formulation of promoting intracellular inflow of the siRNA. The formulation of promoting intracellular inflow of the siRNA may generally use a formulation of promoting nucleic acid inflow, and for example, may use ribosome or be mixed together with one lipophilic carrier of a plurality of sterols including cholesterol, cholate, and deoxycholic acid. Further, the formulation may also use cationic polymers, such as poly-L-lysine, spermine, polysilazane, polyethylenimine (PEI), polydihydroimidazolenium, polyallylamine, and chitosan and use anionic polymers, such as succinylated PLL, a succinylated PEI, polyglutamic acid, polyaspartic acid, polyacrylic acid, polymethacylic acid, dextran sulfate, heparin, and hyaluronic acid. Further, in the case of using a specific antibody to the H2AFZ protein (H2A.Z.1) as the material for decreasing the expression and the activity of the H2A.Z.1 protein, the antibody may be coupled (for example, covalently bound) with an existing therapeutic agent directly or indirectly through a linker. The therapeutic agent which may be bound with the antibody is not limited thereto, but may be bound with radionuclide such as 131I, 90Y, 105Rh, 47Sc, 67Cu, 212Bi, 211At, 67Ga, 1251, 186Re, 188Re, 177Lu, 153Sm, 123I, and 111In; a biologically reactive variant or drug such as methotrexate, adriamycin, and lympokine such as interferon; a toxin such as ricin, abrin, and diphtheria; an antibody in which the complex is bound with heterofunctional antibodies, that is, other antibodies to be bound with both a cancer cell and an efficacy cell (for example, a killer cell (K cell) such as a T cell); and a natural, that is, non-associated or non-conjugated antibody.

Further, in the present disclosure, the pharmaceutical composition may further include a pharmaceutically acceptable carrier. Further, the above “pharmaceutically acceptable” generally means a composition which is physiologically acceptable and does not generally cause an allergic reaction such as gastrointestinal disturbance and dizziness or a similar reaction thereto, when being administrated to the human body. The pharmaceutically acceptable carrier includes, for example, a carrier for oral administration such as lactose, starch, a cellulose derivative, magnesium stearate, and stearic acid and a parenteral administration carrier such as water, suitable oil, saline, aqueous glucose, and glycols, and a stabilizer and a preservative may be further included. The suitable stabilizer includes an antioxidant such as sodium bisulfite, sodium sulfite, or ascorbic acid. The suitable preservative includes benzalkonium chloride, methyl- or propyl-paraben, and chlorobutanol. Other pharmaceutically acceptable carriers may refer to carriers disclosed in the document below (Remington's Pharmaceutical Sciences, 19th ed., Mack Publishing Company, Easton, Pa., 1995).

The pharmaceutical composition according to the present disclosure may be formulated in a suitable form by a known method in the art together with the pharmaceutically acceptable carrier as described above. That is, the pharmaceutical composition according to the present disclosure may be prepared in various parenteral or oral administration forms according to a known method, and a representative formulation for parenteral administration may be an isotonic aqueous solution or suspension as an injection formulation. The injection formulation may be prepared according to a known technique in the art by using a suitable dispersant or wetting agent and a suspension. For example, each ingredient is dissolved in a saline or buffer solution to be formulated for injection. Further, the formulation for oral administration is not limited thereto, but includes powder, granules, tablets, pills and capsules.

The pharmaceutical composition formulated by the above method may be administrated through various routes including oral, transdermal, subcutaneous, intravenous or muscular with an effective dosage, and the administration means introducing a predetermined material to the patient by any suitable method and the administration route of the material may be administrated through a general route so long as reaching a desired tissue.

Further, the effective dosage means an amount representing a preventing or treating effect when being administrated to the patient. The dosage of the pharmaceutical composition according to the present disclosure may vary according to various factors such as a type and severity of disease, age, sex, and weight of a patient, sensitivity to a drug, a type of a current treatment method, an administration method, and a target cell and may be easily determined by experts in the art. Further, the pharmaceutical composition of the present disclosure may be administrated in combination with therapeutic agents in the related art, sequentially or simultaneously administrated with the therapeutic agents in the related art, and administrated in single or multiple. Preferably, a dosage capable of obtaining a maximum effect as a minimal amount without side effects may be administrated by considering all of the elements, and may be repetitively administrated several times per a day as an effective amount of more preferably 1 to 10000 μg/kg/day, and much more preferably 10 to 1000 mg/kg/day.

Hereinafter, the present disclosure will be described in more detail through Examples. However, these Examples are just to exemplify the present disclosure, and it is not interpreted that the scope of the present disclosure is not limited to these Examples.

1. Preparation of Experiment

(1) Preparation of Tissue Sample

16 hepatocellular carcinoma (HCC) tissues and 6 pairs, and a non-tumor cell hepatic tissue corresponding thereto were obtained from a pathology classroom in Yonsei University (Seoul, Korea) and a liver cancer specimen bank of the Korea Science Foundation under the Ministry of Science and Technology (currently, Korea Research Foundation under the Department of Creation Science). According to the Helsinki Declaration, written consents were received from all subject samples and approved by the Investigational Review Committee (IRB) of the Medical Collage (Songeui Campus), Catholic University ORB approval number: MC12SNMI0184).

(2) Cell Culture

Hep3B, HepG2, Huh7, PLC/PRF/5, SK-Hep1, SNU-182, SNU-368, SNU-449 and SNU-475 HCC were obtained from Korea Cell Line Bank (Seoul, Korea). A non-tumor normal hepatocyte line (MIHA) was purchased from ATCC (Virginia, USA). All cell lines were incubated in a DMEM medium (Lonza, Maryland) including RPMI-1640 or 10% fetal bovine serum (Sigma, Missouri) and 100 units/ml of penicillin-streptomycin (Invitrogen, California). All cells were incubated in a humidity incubator including 5% carbon dioxide at 37° C.

(3) Gene Expression Data

In order to analyze expression levels of H2AFZ and H2AFV in the HCC, a gene expression profiling data set was obtained from a gene expression ominibus (GEO) database of the National Center for Biotechnology Information (NCBI) (Accession Nos. GSE14520, GSE16757, GSE22058, GSE29813, GSE35289, GSE36376 GSE57597).

(4) Western Blot Analysis

Cells were lysed in a lysis buffer (50 mM HEPES, 5 mM EDTA, 50 mM sodium chloride, 1% Triton X-100, 50 mM sodium fluoride (NaF), 10 mM Na₂P₄O₇, 1 mM Na₃VO₄, 5 μg/ml aprotinin, 5 μg/ml Leupeptin, 1 mM PMSF and protease inhibitor cocktail). A lysate including the same amount of protein was isolated by SDS-PAGE and transferred to polyvinylidene difluoride membrane (Bio-Rad, California). Blots were blocked in a 5% nonfat milk solution and incubated together with the following antibodies: anti-H2A.Z.1, anti-p21, anti-PARP, anti-caspase-9, anti-caspase-3, anti-cleaved caspase-3, anti-p27, anti-Cyclin D1, anti-Cyclin A2, anti-CDK2, anti-p-pRb, anti-vimentin, anti-slug (Cell Signaling Technology, Massachusetts), anti-GAPDH, anti-Fibronectin (Santa Cruz Biotechnology, California), anti-E-cadherin, anti-N-cadherin (BD Transduction, California) and anti-Snail (Abcam, Massachusetts). The bound antibodies were detected by using an Immobilon™ western blot detection system (Millipore, Massachusetts). Intensity of the western blot was measured by using LAS-3000 (Fuji Photo Film Co., Japan).

(5) Cell Viability Assay

The cells were inoculated in a 6-well dish and infected with a negative control group siRNA or H2A.Z.1 siRNA (5′ GAAGAAAGGACAACAGAAGACT 3′). After infection, the cells were incubated for 72 hrs and collected with trypsin, and then colored with a Trypan-blue solution (Sigma, Missouri). After coloring, the cells were counted by using a hemocytometer (Marienfeld Superior, Germany).

(6) Cell Proliferation Assay

A relative cell proliferation was measured by using a 3-(4,5-dimethylthiazol-2-yl)-5-(3-carboxymethoxyphenyl)-2-(4-sulfophenyl)-2H-tetrazolium (MTS) assay. The cells were put in a 12-well dish and infected with a negative control group siRNA and H2A.Z.1 siRNA. After infection, in order to measure cell proliferation, the cells were measured every 24 hrs by using a 500 μl cellTriter 96 One Solution Cell Proliferation assay solution (Promega, Wisconsin) diluted with 1/20. After one hour, cell absorbance was measured at a wavelength of 490 nm with a VICTOR3™ Multilabel plate reader (PerkinElmer Inc, Massachusetts).

(7) Bromodeoxyuridine (BrdU) Binding Assay

The cells were put in a 24-well dish until 40% to 50% confluence. The cells inoculated with the negative control group siRNA or H2A.Z.1 siRNA were infected and the assay was performed every 24 hrs according to a protocol of a manufacturer by a BrdU cell proliferation as say kit (Millipore, Massachusetts).

(8) Clonogenic Assay

The cells were infected with H2A.Z.1 siRNA in a 60 mm² cell culture dish. After infection for 24 hrs, 2×10³, 4×10³, and 6×10³ cells were re-inoculated in a 6-well dish and incubated for 2 weeks. Thereafter, the cells were washed with a phosphate-buffered saline and immobilized with 1% paraformaldehyde at room temperature for 30 minutes. The immobilized cells were colored with 0.5% crystal violet at room temperature for 1 hr. Colonies were measured by using a colon counter program (Niyazi M, Niyazi I and Belka C. Counting colonies of clonogenic assays by using densitometric software. Radiat Oncol. 2007; 2:4.).

(9) Cell Cycle Assay

In order to determine a cell distribution, the cells were inoculated in a 60 mm² dish and then infected with negative control groups siRNA and H2A.Z.1 siRNA. After incubated for 72 hrs, the cells were collected by trypsin treatment. Thereafter, the cells were immobilized by using 70% ethanol, washed with a phosphate-buffered saline, and then re-suspended with a 200 μl phosphate-buffered saline including 1 mg/ml RNase, 0.05% Triton X-100 and 50 μg/ml propidium iodide (BD Biosciences, California). Thereafter, the suspended cells were incubated in a 37° C. incubator for 1 hr without carbon dioxide and then assayed by using a FACSCalibur flow cytometer (BD Biosciences, California) installed in FLOWJO software (Tree Star, Oregon).

(10) Apoptosis Assay

An apoptosis level was measured by using Annexin V-FITC Apoptosis Detection Kit I (BD Biosciences, California). After infection for 72 hrs, the cells were treated with trypsin, washed with a phosphate-buffered saline, and re-suspended with a 1× binding buffer (1×10⁶ cells/ml), and 100 μl re-suspended solution including 1×10⁵ cells was transferred to a 5 ml incubate tube. Thereafter, 5 μl Annexin V-FITC and a 5 μl propidium iodide (PI) solution were added. The cells were incubated at room temperature for 15 mins in the dark. After incubation, 400 μl of 1× binding buffer was put in each tube, an apoptosis piece was detected by using a FACSCalibur flow cytometer (BD Biosciences, California), and analyzed with FLOWJO software (Tree Star, Oregon).

(11) RNA Isolation and Quantitative Real-Time Reverse Transcription Polymerase Chain Reaction (qRT-PCR)

A total RNA was isolated from frozen tissue and cell line according to a manual of the manufacturer by using a TRIzol reagent (Invitrogen, California). In order to synthesize cDNA, a tetro cDNA synthesis kit (Bioline USA Inc., Massachusetts) was used. For a quantitative real-time reverse transcription polymerase chain reaction (qRT-PCR) assay, the reaction was performed by using a SensiFAST™ SYBR No-ROX kit (Bioline USA Inc., Massachusetts). A GAPDH level was used as a loading control group. The PCR reaction was monitored by using IQ-5 (Bio-Rad) to verify threshold cycle (Ct): a logarithmic amplification time of a PCR product. A relative expression level was standardized by using a control group-2(^{Target Ct- Control Ct}). Primers used in H2AFZ amplification were as follows.

Forward
5′-GCAGTTTGAATCGCGGTG-3′
Reverse
5′-GAGTCCTTTCCAGCCTTACC-3′

GAPDH primers were as follows.

Forward
5′-ACCAGGTGGTCTCCTCTGAC-3′
Reverse
5′-TGCTGTAGCCAAATTCGTTG-3′

(12) Motility and Invasion Assay

For motility and invasion assay in vitro, cell migration was measured by using a modified Boyden chamber assay (BD Bioscience, California). For the invasion assay, a matrigel (BD Biosciences) was diluted with a coating buffer (0.01 M Tris, 0.7% sodium chloride, pH 8.0) at a 0.3 mg/ml concentration. Thereafter, the top of a cell culture insert was coated with 100 μl of the matrigel. An insert inoculation was prepared when incubated for 2 hrs at 37° C. After the insert was prepared, the cells was placed on the upper surface of a transwell insert (8-μm pore size) and the insert was placed in a 24-well dish. A lower well included 5% fetal bovine serum as a chemical injection factor. The dish was incubated overnight and then immobilized by using a Diff-Quick staining kit (Sysmex, Japan). A cell image was photographed at ×200 magnification by using an Axiovert 200 inverted microscope (Zeiss, Germany) and the cell number was counted in three random image fields.

(13) Wound Healing Assay

The cells were infected and then included in a 60 mm² cell culture dish and incubated for 24 hrs. Thereafter, the cells were treated with trypsin and 1×10⁶ cells were inoculated in a 6-well cell culture dish. After inoculated overnight, cell monolayers were scraped by using a sterile micropipette tip. An initial gap width after scrap 0 hr and a residual gap width after scratching 24 hrs were imaged by a microphotograph.

(14) Gene Set Enrichment Analysis (GSEA)

In order to examine a basic mechanism of the H2A.Z.1 gene, a gene set enrichment analysis (GSEA) was performed by using a H2AFZ relation gene set defined in a large-scale Cohort study (GSE16757). With respect to a gene data set ranked by relation with gene expression levels by interest phenotypes, a basic GSEA analysis provided scores quantifying the degree of concentration of a given gene set in an upper rank (positive relation) or a lower rank (negative relation) of the ranked data set. Proximity of the gene set was measured by Kolmogorov-Smirnoff (KS) scores (a higher score indicates greater proximity). The observed KS score was compared with a 1000 substituted KS score distribution with respect to all gene sets in order to evaluate significance.

(15) Molecular Concept Map

A network graph illustrating interconnection between genetic sets was created by using an open source gene set enrichment testing and ConceptGen which was concept diagram creating tool (http://conceptgen.ncibi.org/core/conceptGen/index.jsp). In order to provide a functional relation with a H2AFZ specific gene set in HCC, with respect to a H2AFZ relation gene set defined in a large-scale Cohort study (GSE16757), a molecular relation was evaluated by using a ConceptGen Molecular Concept Map (MCM) including 20,000 or more molecules consisting of 14 biological knowledge types and imbalance overlap was adjusted by using a modified Fisher's extract test (Sartor M A, Mahavisno V, Keshamouni V G, Cavalcoli J, Wright Z, Karnovsky A, Kuick R, Jagadish H V, Mirel B, Weymouth T, Athey B and Omenn G S. ConceptGen: a gene set enrichment and gene set relation mapping tool. Bioinformatics. 2010; 26(4):456-463). With respect to 477 H2AFZ correlated genes (p<0.01, r>0.4 or r<−0.4) and other tumor-related genes, an MCM assay was performed and an enrichment network was created.

(16) Statistical Processing

All experiments were performed at least three times and all samples were analyzed three times. A result value was recorded with mean±standard deviation (SD). A statistical difference between experimental groups was evaluated by performing an unpaired two-tailed Student's t-test with Graphpad™ 5.0 (GraphPad software Inc., California). When a P value was 0.05 or less, a statistical significance was exhibited.

2. Experimental Result

(1) in an HCC patient, an H2A.Z.1 gene was abnormally overexpressed and the overexpression was involved with bad prognosis of the HCC.

In order to evaluate H2A.Z expression in the HCC, in the large-scale HCC Cohort study (accession numbers GSE14520, GSE16757, GSE22058 and GSE36376) affordable from the National Center for Biotechnology Information (NCBI) and a Gene Expression Omnibus (GEO) database, H2AFZ and H2AFV expression was summarized.

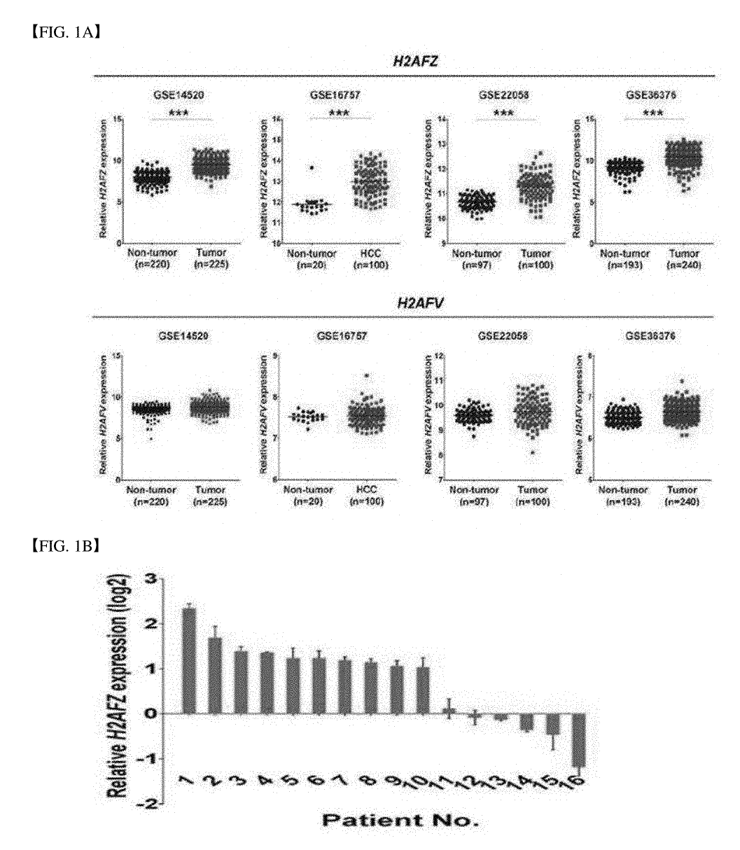

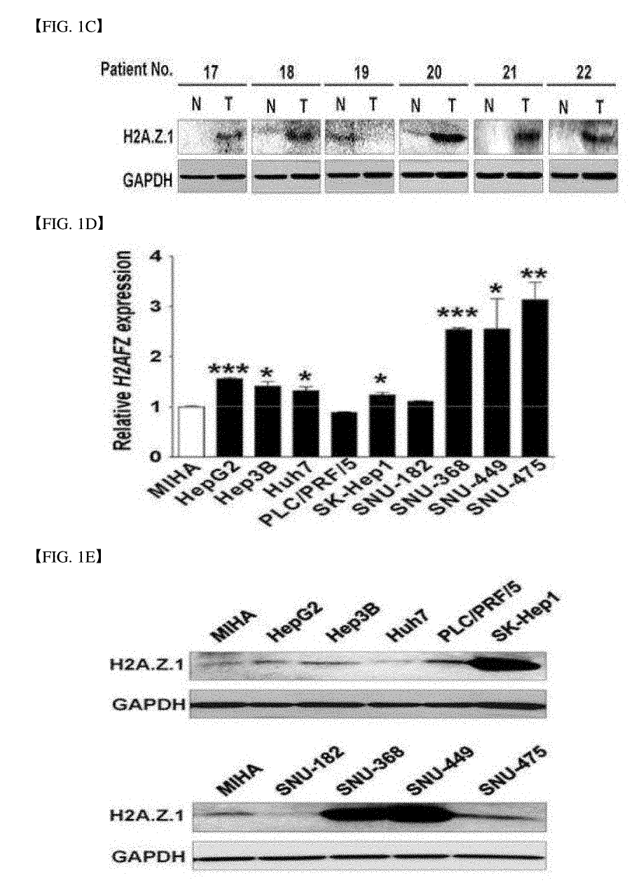

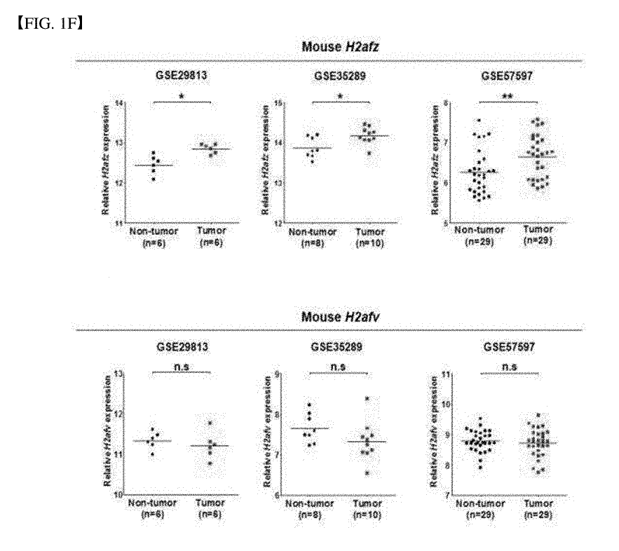

Unlike metastatic melanoma, in four HCC Cohort studies, only the H2AFZ gene was significantly overexpressed, whereas the expression of the H2AFV gene was not changed (see FIG. 1A).

In order to verify overexpression of the H2AFZ gene in the HCC patient, gene expression of H2AFZ in 16 randomly extracted HCC cell tissues made a pair with an adjacent non-tumor liver tissue to perform a quantitative real-time-PCR (qRT-PCR). As the experimental result, in a total of 10 tissues of 16 HCC cell tissues, the H2AFZ gene was overexpressed (FIG. 1B).

As a result of performing an immune blot for 6 randomly selected HCC cell tissues and a non-tumor liver tissue corresponding thereto, it was verified that expression of the H2A.Z.1 protein was increased like the result (FIG. 1C).

Further, with respect to 10 different liver cell lines including a non-tumor normal liver cell line (MIHA), the qRT-PCR was performed and intrinsic expression of H2AFZ was studied (FIG. 1D). The human HCC cell lines (HepG2, Hep3B, Huh7, SK-Hep-1, SNU-368, SNU-499 and SNU-475) had a relatively higher H2AFZ gene expression level than the non-tumor normal liver cell line (MIHA). The human HCC cell lines also had a relatively higher H2AFZ protein expression level than the non-tumor normal liver cell line (MIHA) (FIG. 1E).

Even in an animal experiment, in three different mouse HCC studies (accession numbers GSE29813, GSE35289, and GSE57597) which may be obtained in NCBI and GEO databases, H1AFZ and H2AFV expression was summarized. Like the result in the human, in a mouse HCC GEO data set, only the H2AFZ was significantly overexpressed in the mouse HCC (see FIG. 1F).

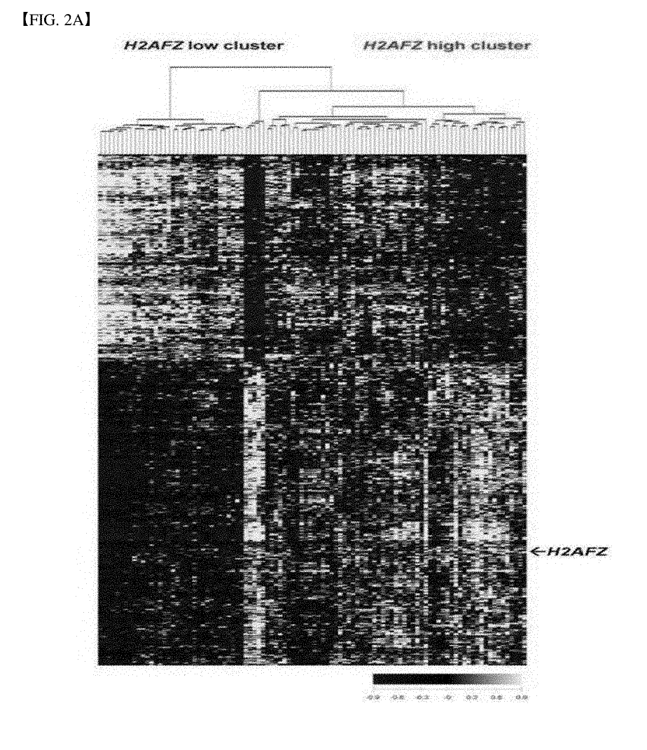

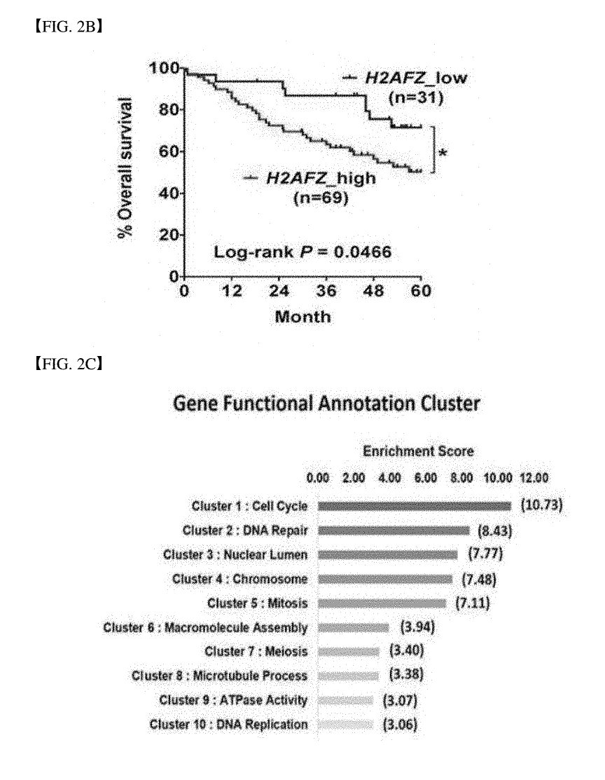

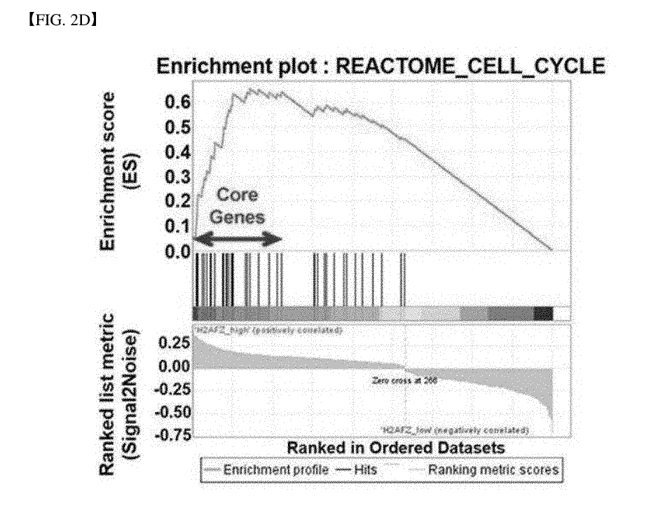

Because it was verified that mRNA and protein levels of H2A.Z.1 were increased, in a large-scale Cohort study (GSE16757) for 100 HCC patients, the prognostic relevance of H2A.Z.1 expression was evaluated. 524 genes having expression tendency with a high relation with H2A.Z.1 expression were selected for a cluster analysis (P<O/001, r>0.4 or r<−0.4) and illustrated with Heatmaps (FIG. 2A). Thereafter, the patients were classified into an H2A.Z.1 high cluster group (H2AFZ_high) and an H2A.Z.1 low cluster group (H2AFZ_low). According to a Kaplan-Meier survival curve for HCC patient groups, 5-year mean survival rate in an H2A.Z.1 high expression group was significantly lower than the survival rate of an H2A.Z.1 low expression group (P=0.0466, FIG. 2B). The result means that in the HCC group, the H2A.Z.1 expression is increased in HCC patient groups and the high expression thereof is associated with tumor formation and bad disease prognosis of the HCC patient.

In order to study a biological relevance of a H2AFZ-related molecular marker, the gene cluster (a total of 524 genes) was input to a DAVID Web-based bioinformatics platform (http://david.abcc.ncifcrf.gov/) to retrieve a biological route of a H2AFZ indicator. As a result, gene functional annotation cluster mapping generated by a part of the DAVID tool exhibited a biological process which discovers the molecular biological data set (see FIG. 2C). Main signal pathways of the H2AFZ index include a cell cycle, a DNA repair, a nuclear lumen, a chromosome, a mitosis, a macromolecule assembly, a meiosis, a microtubule process, ATPase activity and DNA replication (see FIG. 2C and Table 1).

TABLE 1

| Cluster No. | Title | Term | Count | p value |
|---|---|---|---|---|
| 1 | Cell Cycle | cell cycle phase | 45 | p < 0.001 |
| | | cell cycle | 58 | p < 0.001 |
| | | M phase | 35 | p < 0.001 |
| | | cell cycle process | 47 | p < 0.001 |
| | | mitotic cell cycle | 35 | p < 0.001 |
| | | organele fasion | 35 | p < 0.001 |
| | | mitosis | 24 | p < 0.001 |
| | | nuclear division | 24 | p < 0.001 |
| | | M phase of mitosis cell cycle | 24 | p < 0.001 |
| | | cell division | 23 | p < 0.001 |
| 2 | DNA Repair | DNA metabolic process | 45 | p < 0.001 |
| | | DNA repair | 25 | p < 0.001 |
| | | response to DNA damage stimulus | 35 | p < 0.001 |
| | | cellular response to stress | 33 | p < 0.001 |
| 3 | Nuclear Lumen | organelle lumen | 52 | p < 0.001 |
| | | nucleoplasm | 51 | p < 0.001 |
| | | intracellular organelle lumen | 88 | p < 0.001 |
| | | membrane-enclosed lumen | 82 | p < 0.001 |
| | | nuclear lumen | 66 | p < 0.001 |
| 4 | Chromosome | chromosome | 44 | p < 0.001 |
| | | chromosome part | 27 | p < 0.001 |
| | | condensed chromosome | 23 | p < 0.001 |
| | | chromosome centromeric region | 18 | p < 0.001 |
| | | kinetochore | 12 | p < 0.001 |
| | | condensed chromosome, centromeric region | 12 | p < 0.001 |
| | | condensed chromosome, kinetochore | 11 | p < 0.001 |
| | | chromosome segregation | 11 | p < 0.001 |
| | | non-membrane-bounded organelle | 55 | p < 0.001 |
| | | intracellular non-membrane-bounded organelle | 55 | p < 0.001 |
| 5 | Mitosis | mitosis cell cycle | 35 | p < 0.001 |
| | | intraphase of mitosis cell cycle | 15 | p < 0.001 |
| | | interphase | 15 | p < 0.001 |
| | | G1/5 transition of meiotic cell cycle | 5 | p < 0.001 |
| 6 | Macromolecule | macromolecular complex subunit organization | 39 | p < 0.001 |
| | Assembly | macromolecular complex assembly | 35 | p < 0.001 |
| | | cellular macromolecule complex subunit organization | 23 | p < 0.001 |
| | | cellular macromolecular complex assembly | 19 | p < 0.001 |
| | | protein complex biogenesis | 25 | p < 0.01 |
| | | protein complex assembly | 25 | p < 0.01 |
| 7 | Meiosis | DNA recombination | 13 | p < 0.001 |
| | | meiotic cell cycle | 12 | p < 0.001 |
| | | M phase of meiotic cell cycle | 11 | p < 0.001 |
| | | meiosis | 11 | p < 0.001 |
| | | reciprocal meiotic recombination | 4 | p < 0.05 |
| | | meosis 1 | 4 | n.s. |
| 8 | Microtubule | microtubule cytoskeleton organization | 14 | p < 0.001 |
| | Process | spindle organization | 8 | p < 0.001 |
| | | microtubule-based process | 17 | p < 0.001 |
| | | cytoskeleton organization | 19 | p < 0.05 |
| 9 | ATPase Activity | DNA-dependent ATPase activity | 9 | p < 0.001 |
| | | ATPase activity | 33 | p < 0.001 |
| | | ATPase activity, couples | 15 | p < 0.01 |
| 10 | DNA Replication | replication fork | 5 | p < 0.001 |
| | | DNA replication factor C complex | 4 | p < 0.001 |
| | | nucleotide-excision repair, DNA gap filling | 5 | p < 0.001 |
| | | DNA clamp loader activity | 3 | p < 0.001 |
| | | protein-DNA loading ATPase activity | 3 | p < 0.01 |
| | | nucleotide-excision repair | 5 | p < 0.01 |

In order to more understand a molecular mechanism of H2A.Z.1 in HCC occurrence, a gene set enrichment analysis (GSEA) was performed by using the H2AFZ index in order to define the H2AFZ-related gene set. The GSEA defined an intracellular signal pathway of the H2AFZ index in the HCC cells and REACTOME_CELL_CYCLE was a gene which was significantly high-concentrated in the H2AFZ index (see FIG. 2D and Table 2).

TABLE 2

| No. | Gene Sets | SIZE | ES | NES |
|---|---|---|---|---|
| 1 | REACTOME_CELL_CYCLE | 42 | 0.65 | 1.35 |
| 2 | REACTOME_CELL_CYCLE_MITOTIC | 38 | 0.64 | 1.32 |
| 3 | REACTOME_MITOTIC_M_M_G1_PHASES | 23 | 0.66 | 1.28 |
| 4 | REACTOME_MITOTIC_PROMETAPHASE | 16 | 0.62 | 1.24 |
| 5 | REACTOME_DNA_REPLICATION | 28 | 0.6 | 1.2 |
| 6 | REACTOME_ADAPTIVE_IMMUNE_SYSTEM | 17 | 0.4 | 1.16 |
| 7 | REACTOME_IMMUNE_SYSTEM | 25 | 0.24 | 0.76 |
| 8 | REACTOME_HEMOSTASIS | 22 | 0.18 | 0.55 |
| 9 | REACTOME_METABOLISM_OF_LIPIDS_AND_LIPOPROTEINS | 16 | 0.19 | 0.53 |

(2) H2A.Z.1 target inhibition caused cancer inhibition action through cell cycle and EMT protein regulation in the HCC cells.

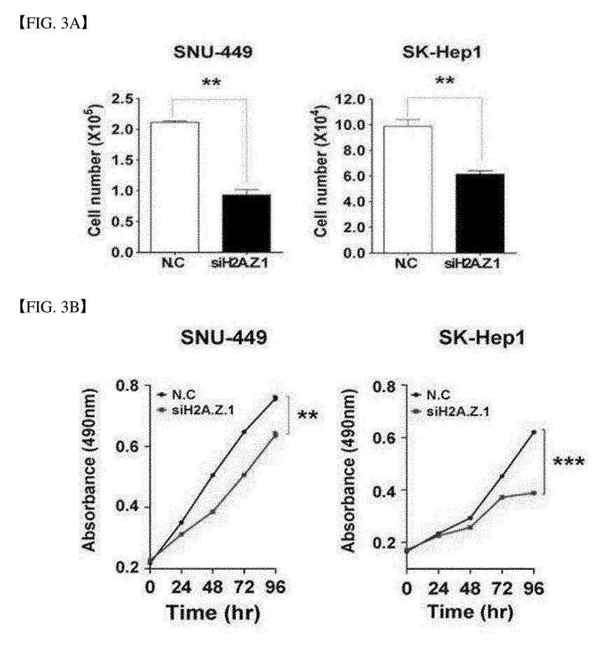

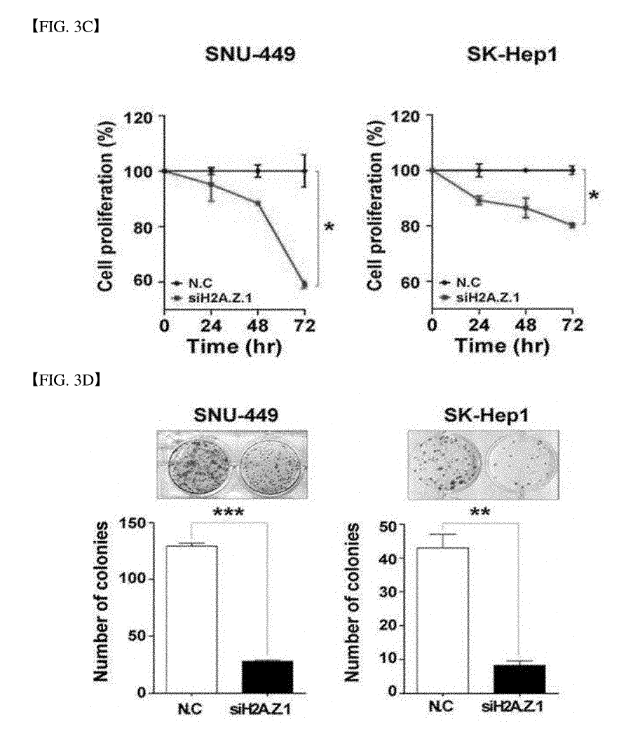

In order to more understand a molecular function of H2A.Z.1 in the liver tumor occurrence, H2A.Z.1 knock-down was attempted by an RNA interference method and cell viability, MTS cell proliferation, BrdU binding and clonogenic assay were performed. During the H2A.Z.1 knock-down, in SNU-449 and SK-Hep1 HCC cells, growth and proliferation speeds were reduced (see FIGS. 3A to 3D).

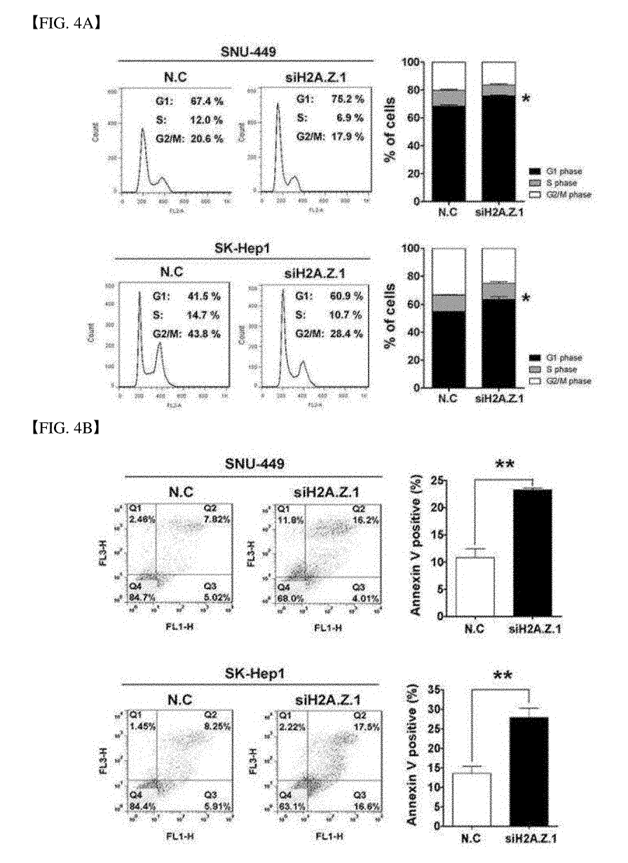

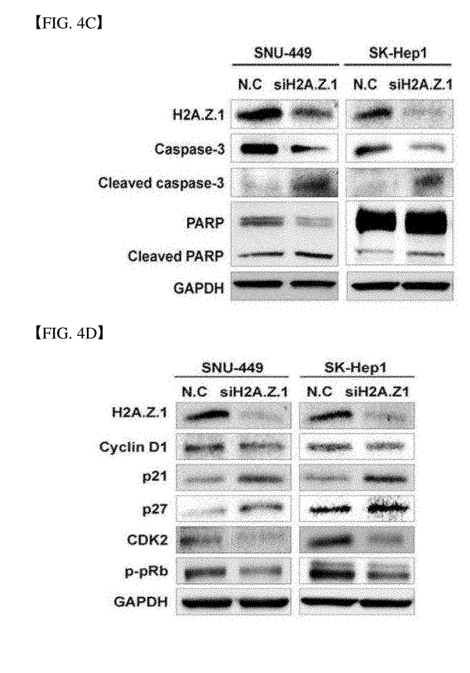

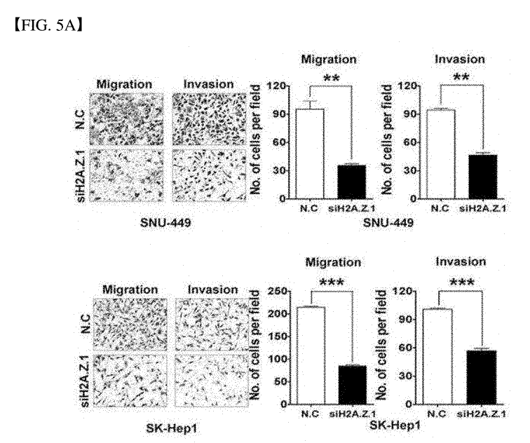

The growth inhibition effect may be partially described by division of cell growth regulation such as cell cycle arrest, cellular senescence, and apoptosis targeting H2A.Z.1. According to a flow cell cycle analysis, it was exhibited that during the H2A.Z.1 knock-down, in both SNU-499 and SK-Hep1 HCC cell lines, the cell number in a G1 phase was significantly increased and accordingly, the cell numbers in S and G2/M phases were decreased (FIG. 4A).

For apoptosis analysis, the cells were infected with H2A.Z.1 target siRNA (siH2A.Z.1) and then stained with annexin V-FITC and PI. During the H2A.Z.1 knock-down, in SNU-449 and SK-Hep1 HCC cell lines, the apoptosis cell number was significantly increased compared with a negative control group siRNA (N.C) and capsage-3 and PARP cleavages were increased (see FIGS. 4B and 4C).

Further, since the GSEA defined REACTOME_CELL_CYCLE as a significantly high-concentrated gene in the H2AFZ index, a growth inhibition mechanism caused by an H2A.Z.1 target was clarified by performing a western blot assay for a cell cycle protein. As the western blot assay result, during the H2A.Z.1 knock-down, in both SNU-449 and SK-Hep-1 cells, expression of cyclin D1 and cyclin A2 was inhibited and expression of p21^{WAFl/CiPa} and P27^{Kipl} was increased (FIG. 4D). The result illustrates that a specific destruction of the H2A.Z.1 gene inhibited a cell cycle negative regulator such as p21^{WAFl/CiPa} a and P27^{Kipl} and simultaneously, increased a specific regulator such as cyclin D or CDK2.

Generally, the activated cyclin/CDK complex causes hyperphosphorylation of pRb to loss tumor suppressive ability thereof and increase E2F/DP1 transcription activity. Accordingly, according to cyclin and CDKs reduction by an H2A.Z.1 change, it is evaluated whether the E2F/DP1 transcription activity was changed. As expected, during H2A.Z.1 targeted destruction, in both SNU-449 and SK-Hep-1 cells, the hypophosphorylation of pRb was caused. As a result, specific regulation for H2A.Z.1 means influencing phosphorylation of the Rb protein and inactivation of an HCC formation inhibitor through the transcription activity of cyclin/CDK.

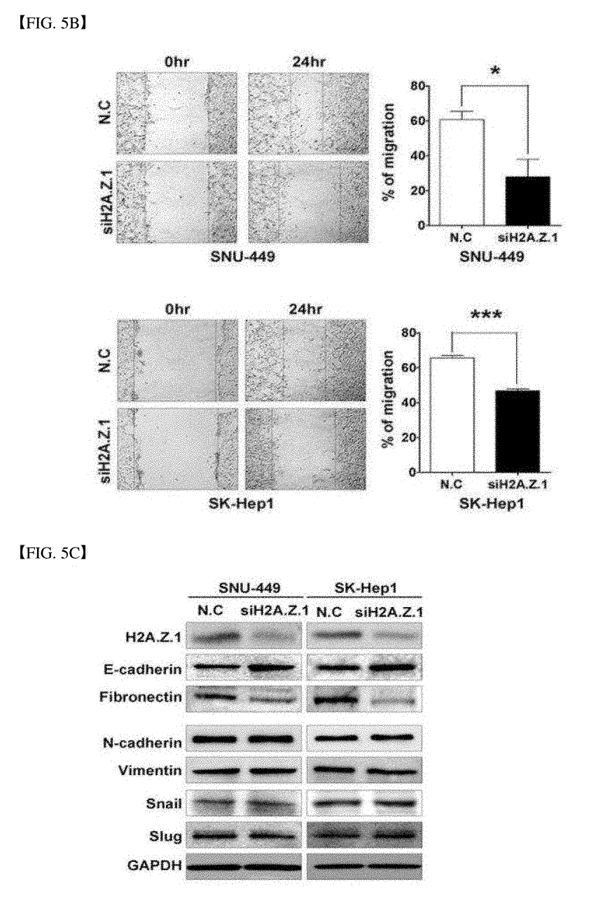

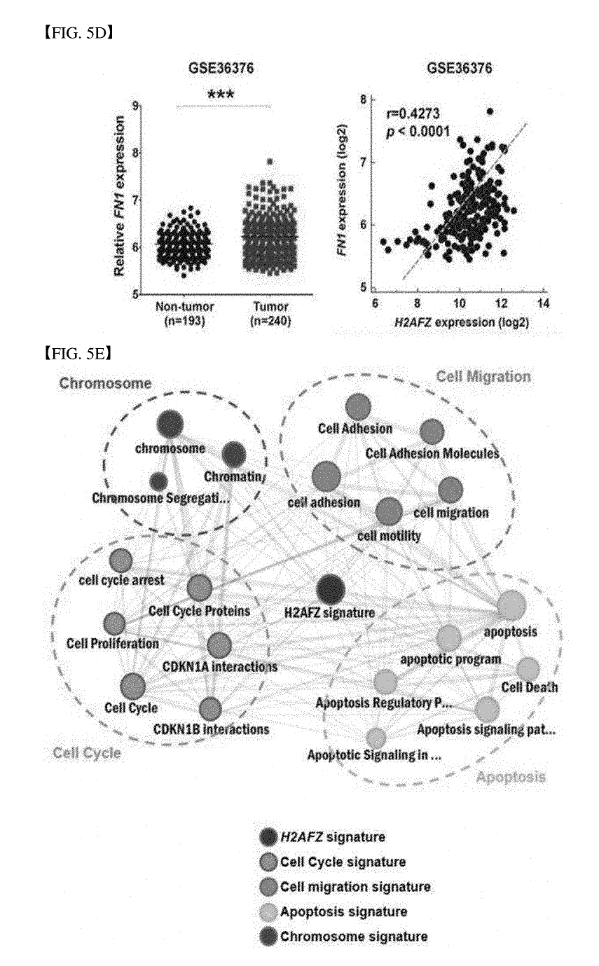

Further, the H2A.Z.1 index was analyzed by integrating all tumor-related gene expression indexes and using a molecular concept diagram (ConceptGen) (Non-Patent Document 4). Through the analysis, elements associated with H2A.Z.1-related gene elements is described, elements of associating a cell cycle, cell migration, apoptosis and a chromosomal control program associating with the H2A.Z.1 index were defined (see FIG. 5E).

As a result, it was seen that the H2A.Z.1 index was highly related to cell migration or a cell migration associated index.

In order to define a function in the HCC cells of the H2A.Z.1 gene, motility and invasion analysis in vitro was performed. Through the modified Boyden chamber analysis, it was verified that during the H2A.Z.1 knock-down, in both SNU-449 and SK-Hep-1 cells, cell migration and invasive reaction induced by a chemoattractant (5% fetal bovine serum) were significantly reduced (FIG. 5A). Further, through a scratch wound healing assay, it was verified that during the H2A.Z.1 knock-down, wound healing efficiency induced by the chemoattractant (5% fetal bovine serum) was reduced (FIG. 5B).

In order to verify an effect of H2A.Z.1 on epithelial-mesenchymal transition (EMT), a western blot assay for an EMT protein was performed. Surprisingly, in both the SNU-449 and SK-Hep-1 cells, fibronectin as a typical feature of the EMT was decreased in an siH2A.Z.1 infectious agent, whereas E-cadherin as an epidermal index was increased (FIG. 5C).

Further, even in the gene expression analysis of the large-scale HCC Cohort study (accession number GSE36376) obtained from an NCBI GEO database, the fibronectin gene expression was significantly increased in an HCC group and a positive relation was exhibited (FIG. 5D).

The results mean that metastasis of the H2A.Z.1 gene is caused by selective regulation for the EMT protein such as fibronectin and E-cadherin in the HCC cells.

Although the specific part of the present disclosure has been described in detail, it is obvious to those skilled in the art that such a specific description is just a preferred embodiment and the scope of the present disclosure is not limited thereby. Thus, the substantial scope of the present disclosure will be defined by the appended claims and equivalents thereof.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

1. A marker for diagnosing hepatocellular carcinoma (HCC) consisting of an H2A.Z.1 gene or an H2A.Z.1 protein encoded from the gene.

2. The marker for diagnosing HCC of claim 1, wherein the H2A.Z.1 gene has a base sequence of SEQ ID NO: 1.

3. A composition for diagnosing or estimating prognosis of HCC including a material for measuring an expression level of an H2A.Z.1 gene of claim 1.

4. The composition for diagnosing or estimating prognosis of HCC of claim 3, wherein the material for measuring the expression level of the gene is a material that detects at least one of presence, amount, and abundance pattern of mRNA transcribed by the gene and/or a protein encoded by the gene.

5. The composition for diagnosing or estimating prognosis of HCC of claim 3, wherein the material for measuring the expression level of the gene is at least one of a primer, a probe, an aptamer, and an antisense which are specifically bound to at least one selected from the group consisting of a nucleotide sequence of the gene, a complementary sequence thereof, a fragment of the nucleotide and a complementary sequence thereof.

6. The composition for diagnosing or estimating prognosis of HCC of claim 3, wherein the material for measuring the expression level of the gene is at least one selected from oligopeptides, monoclonal antibodies, polyclonal antibodies, chimeric antibodies, antibody fragments, ligands, peptide nucleic acids (PNA), aptamers, avidity multimers, and peptidomimetics which specifically bind to at least one of a polypeptide encoded by a nucleotide sequence of the gene, a polypeptide encoded by a complementary sequence thereto, and a polypeptide encoded by a fragment of the nucleotide sequence.

7. The composition for diagnosing or estimating prognosis of HCC of claim 3, wherein the material for measuring the expression level of the gene is a detection reagent of measuring a gene expression by at least one method of a reverse transcription polymerase chain reaction, a competitive polymerase chain reaction, a real-time polymerase chain reaction, a nuclease protection assay (RNase, S1 nuclease assay), an in situ hybridization method, a DNA microarray method, northern blotting, western blotting, an enzyme linked immuno sorbent assay (ELISA), a radioimmunoassay, an immunodiffusion method, immunoelectrophoresis, a tissue immuno staining, an immunoprecipitation assay, a complement fixation assay, an FACS, a mass spectrometry and a protein microarray.

8. A kit for diagnosing or estimating prognosis of HCC including the composition for diagnosing or estimating prognosis of the HCC according to claim 3.

9. The kit for diagnosing or estimating prognosis of HCC of claim 8, wherein the kit is at least one selected from a microarray, a gene amplification kit, an immunoassay kit, a luminex assay kit, a protein microarray kit, and an ELISA kit.

10. A method for diagnosing or estimating prognosis of HCC, comprising: treating a target biological sample with the composition for diagnosing or estimating prognosis of the HCC according to claim 3; measuring an expression level of an H2A.Z.1 gene from the target biological sample; and comparing a measured result of the gene expression level with a reference value.

11. The method for diagnosing or estimating prognosis of HCC of claim 10, wherein the biological sample is selected from the group consisting of tissues, cells, blood, serum, plasma, saliva and urine.

12. The method for diagnosing or estimating prognosis of HCC of claim 10, wherein the measuring of the expression level of the gene uses at least one method of a reverse transcription polymerase chain reaction, a competitive polymerase chain reaction, a real-time polymerase chain reaction, a nuclease protection assay (RNase, S1 nuclease assay), an in situ hybridization method, a DNA microarray method, northern blotting, western blotting, an enzyme linked immuno sorbent assay (ELISA), a radioimmunoassay, an immunodiffusion method, immunoelectrophoresis, a tissue immuno staining, an immunoprecipitation assay, a complement fixation assay, an FACS, a mass spectrometry and a protein microarray.

13. The method for diagnosing or estimating prognosis of HCC of claim 10, further comprising: determining that the HCC is caused when the expression of the H2A.Z.1 gene is increased and the expression of a H2A.Z.2 is not changed.

14. A method of detecting a biomarker for diagnosing or estimating prognosis of HCC through measuring an expression level of an H2A.Z.1 gene of claim 1 in a human biological sample in order to provide information required for diagnosing or estimating prognosis of the HCC.

15. The method of detecting a biomarker for diagnosing or estimating prognosis of HCC of claim 14, wherein the measuring of the expression level of the gene uses at least one method of a reverse transcription polymerase chain reaction, a competitive polymerase chain reaction, a real-time polymerase chain reaction, a nuclease protection assay (RNase, S1 nuclease assay), an in situ hybridization method, a DNA microarray method, northern blotting, western blotting, an enzyme linked immuno sorbent assay (ELISA), a radioimmunoassay, an immunodiffusion method, immunoelectrophoresis, a tissue immuno staining, an immunoprecipitation assay, a complement fixation assay, an FACS, a mass spectrometry and a protein microarray.

16. A screening method of an HCC therapeutic agent including verifying whether a test target compound promotes or inhibits expression of an H2A.Z.1 gene.

17. The screening method of an HCC therapeutic agent of claim 16, wherein the verifying of whether to promote or inhibit the expression of the gene uses at least one method of a reverse transcription polymerase chain reaction, a competitive polymerase chain reaction, a real-time polymerase chain reaction, a nuclease protection assay (RNase, S1 nuclease assay), an in situ hybridization method, a DNA microarray method, northern blotting, western blotting, an enzyme linked immuno sorbent assay (ELISA), a radioimmunoassay, an immunodiffusion method, immunoelectrophoresis, a tissue immuno staining, an immunoprecipitation assay, a complement fixation assay, an FACS, a mass spectrometry and a protein microarray.

18. A pharmaceutical composition for preventing or treating HCC including, as an active ingredient, an antisense or small interference RNA (siRNA) oligonucleotide having a complementary sequence to a base sequence of an H2A.Z.1 gene.

19. The pharmaceutical composition for preventing or treating HCC of claim 18, wherein the siRNA oligonucleotide consists of a base sequence of SEQ ID NO: 2.
